(19) United States
(12) Patent Application Publication
(10) Pub. No.: US 2015/0246232 A1
(43) Pub. Date: Sep. 3, 2015

(54) STIMULUS GENERATOR, A NEUROPROSTHETIC APPARATUS AND A STIMULATION METHOD

(71) Applicants: THE UNIVERSITY OF MELBOURNE, Melbourne, Victoria (AU); NATIONAL ICT AUSTRALIA LIMITED, Eveleigh, New South Wales (AU)

(72) Inventors: Tatiana Kameneva, Melbourne (AU); Hamish Meffin, Melbourne (AU); Dragan Nesic, Melbourne (AU); David Bruce Grayden, Melbourne (AU); Anthony Neville Burkitt, Melbourne (AU)

(21) Appl. No.: 14/411,056

(22) PCT Filed: Jun. 25, 2013
(86) PCT No.: PCT/AU2013/000678

§ 371 Date: Dec. 23, 2014

Related U.S. Application Data
(60) Provisional application No. 61/664,809, filed on Jun. 27, 2012.

(30) Foreign Application Priority Data
Jun. 25, 2012 (AU) 2012902675

(51) Int. Cl. A61N 1/36; A61N 1/05

(52) U.S. Cl. CPC A61N 1/36128; A61N 1/0551; A61N 1/36046

(57) ABSTRACT

A stimulus generator arranged to generate an electrical stimulus to be applied by one or more electrodes to stimulate one or more neurons, wherein in order to generate the stimulus, the stimulus generator: implements one or more neural models defining estimated dynamic behaviour of the one or more neurons; and employs a feedback signal indicative of at least one modelled response of the one or more neurons to at least one previously applied stimulus.

### FIELD

The invention relates to a stimulus generator, a neuroprosthetic apparatus and a stimulation method.

### BACKGROUND

Electrical stimulation of neural tissue has been used to restore function to visually impaired people and people with hearing loss via the implantation of bionic devices. Neuroprosthetic and neuromodulation devices are also used for rehabilitation and treatment of neurological disorders such as epilepsy and Parkinson's disease. In current devices, a significant amount of time is spent on optimizing stimulation parameters post-operatively.

Neural signals have been used for command control and feedback in some medical applications but without resulting in techniques with wide application. For example, feedback protocols are used in paraplegic subjects, to control functionality of artificial limbs, for pain control stimulation and to control peristalsis. Functional electrical stimulation is also available as a clinical tool in muscle activation used for picking up objects, for standing and walking, for controlling bladder emptying, and for breathing. While feedback for functional electrical stimulation has been used in such techniques, to date, such techniques do not have wide application. For example, there is still a need to develop feedback protocols for neuroprosthetic stimulation in auditory and visual prostheses, and the suppression of collective synchrony of neurons in epilepsy and Parkinson's patients.

Accordingly, there is a need for further neuroprosthetic stimulation techniques.

### SUMMARY

In one form, the invention provides a stimulus generator arranged to generate an electrical stimulus to be applied by one or more electrodes to stimulate one or more neurons, wherein in order to generate the stimulus, the stimulus generator:
implements one or more neural models defining estimated dynamic behaviour of the one or more neurons; and
employs a feedback signal indicative of at least one modelled response of the one or more neurons to at least one previously applied stimulus.

In an embodiment, the neural model is based on a spike triggered average response of neurons to stimulus and incorporates a spike history response.

In an embodiment an output of the neural model depends on at least one response of one or more neurons in a time period preceding a time period in which the current stimulus is being generated.

In an embodiment, the output of the neural model depends on a stimulus applied in a time period preceding a time period in which the current stimulus is being generated.

In an embodiment, the stimulus generator comprises a reference signal generator arranged to generate a reference signal to be employed in generating the stimulus.

In an embodiment, a neural modelling component of the stimulus generator implements the neural model defining estimated dynamic behaviour of the one or more neurons, and the reference signal generator also implements a neural model defining estimated dynamic behaviour of the one or more neurons in response to sensory stimulus.

In an embodiment, the sensory stimulus is a visual stimulus.

In an embodiment, the neural model is based on a spike-triggered average response of neurons to stimulus.

In an embodiment, a neural modelling component of the stimulus generator implements the neural model defining estimated dynamic behaviour of the one or more neurons, and the reference signal generator implements a different type of neural model to generate the reference signal.

In an embodiment, a neural modelling component of the stimulus generator implements the neural model defining estimated dynamic behaviour of the one or more neurons, and the reference signal generator implements a different type of neural model to generate the reference signal.

In an embodiment, the estimated dynamic behaviour is based on estimated dynamic behaviour of normally functioning neurons.

In an embodiment, the stimulus generator comprises a reference signal generator arranged to generate a reference signal to be employed in generating the stimulus, the reference signal corresponding to the expected response of normally functioning neurons.

In an embodiment, a reference signal is calculated for each electrode.

In an embodiment, the stimulus generator comprises a static observer that provides an estimate of the internal state of the model on the basis of the stimulus and measured response of one or more neurons.

In an embodiment, the stimulus generator comprises a dynamic observer that provides an estimate of the internal state of the model on the basis of the stimulus and measured response of one or more neurons.

In an embodiment, the feedback signal is based on at least one prior response of at least one of the one or more neurons to a previously applied stimulus.

In an embodiment, the feedback signal is based on at least one prior response of one or more related neurons to a previously applied stimulus.

In another form, the invention provides a neuroprosthetic apparatus comprising the stimulus generator as described above and a plurality of electrodes for applying the generated stimulus and measuring the response of one or more neurons.

In an embodiment, the plurality of electrodes comprise separate stimulation and measurement electrodes.

In an embodiment, the neuroprosthetic apparatus comprises at least one input device for obtaining an external stimulus.

In another form, the invention provides a stimulus generator arranged to generate an electrical stimulus to be applied by one or more electrodes to stimulate one or more neurons, the stimulus generator comprising:
a controller arranged to generate the electrical stimulus; and
a neural modelling component that implements one or more neural models defining estimated dynamic behaviour of the one or more neurons to generate a modelled response indicative of at least one response of the one or more neurons to the generated stimulus; and
wherein the stimulus generator is arranged such that a feedback signal is provided to the controller based on the modelled response, and the controller generates a subsequent electrical stimulus based on the feedback signal.

In an embodiment, the stimulus generator comprises an observer that receives and processes the modelled response and the generated electrical stimulus in order to generate the feedback signal.

In another form, the invention provides a stimulation method for generating an electrical stimulus to be applied in order to stimulate one or more neurons, the method comprising implementing a neural response model defining estimated dynamics of one or more neurons, and adjusting the stimulus to be applied based on a feedback signal indicative of at least one prior response of the one or more neurons to at least one previously applied stimulus.

In another form, the invention provides a stimulation method for generating an electrical stimulus to be applied by one or more electrodes, to stimulate one or more neurons, the method comprising:
using a neural response model defining estimated dynamics of one or more neurons to determine at least one modelled response of the one or more neurons to at least one previously applied electrical stimulus; and
generating a current electrical stimulus to be applied based on a feedback signal indicative of the at least one modelled prior response.

In another form, the invention provides computer program code for implementing one or both of the above methods.

In another form, the invention provides a tangible computer readable medium comprising the computer program code.

Thus, it will be appreciated that embodiments of the invention allow stimulation parameters to be adjusted dynamically, based on the response of neural tissue. That is, embodiments employ advanced engineering techniques, such as feedback control, that allow constant monitoring of the response of neural tissue and optimization of stimulation parameters on-line based on the acquired data. It will also be appreciated that embodiments of the invention enable the provision of a customizable controller in a bionic device thereby providing both scalability and flexibility in manipulating the specific patient-based neural response.

### BRIEF DESCRIPTION OF DRAWINGS

Embodiments of the invention will now be described by way of example with reference to the accompanying drawings in which:

FIG. 1 is a block diagram of a feedback system with external stimulus;

FIG. 2 is a block diagram of a feedback system without external stimulus;

FIG. 3 shows a neuroprosthetic apparatus of an embodiment;

FIG. 4 is a schematic perspective diagram of a four by four electrode array above tissue;

FIG. 5 is a plan view of the electrode array of FIG. 4;

FIG. 6 shows an example of a bi-phasic pulse train;

FIG. 7 shows an example of Gaussian white noise pulse stimulation;

FIG. 8 is a Kalman decomposition of a general system;

FIG. 9 is a Kalman decomposition of a system with the observable/not controllable and controllable/not observable parts of the system set to zero;

FIG. 10 is a diagram of a feedback system of one embodiment;

FIG. 11 shows examples of electrode and neuron reference signals;

FIG. 12 is a block diagram of a feedback system with a reference signal;

FIG. 13 is a block diagram of a filter design;

FIG. 14 is a block diagram of a feedback system including the filter of FIG. 13;

FIG. 15 shows the evoked responses of retinal ganglion cells;

FIG. 16 shows a linear impulse response kernal of the model using reverse-correlation analysis at 25 Hz, 50 Hz, 100 Hz and 200 Hz;

FIG. 17 shows a static nonlinearity using reverse correlation analysis at 25 Hz, 50 Hz, 100 Hz and 200 Hz;

FIG. 18 shows model predictions vs. recorded spike trains for 25 Hz, 50 Hz, 100 Hz and 200 Hz;

FIG. 19 shows the auto-correlation function of the recorded spike trains;

FIG. 20 is a state-space representation of the feedback system;

FIG. 21 is a comparison of the output of systems of three embodiments;

FIGS. 22 and 23 show that the system of various embodiments respond in the same way; and

FIG. 24 shows the evolutions of the systems of various embodiments;

FIG. 25 shows an experimental set up;

FIG. 26 is a block diagram of the system with a static observer;

FIG. 27 shows a reference image (left) and the output of the system with the static observer (right);

FIG. 28 is a block diagram of the dynamic observer;

FIG. 29 is a block diagram of the feedback system with the dynamic observer;

FIG. 30 shows a reference image (left) and the state of the system with the static observer (right);

FIG. 31 shows a reference image (left) and the state of the system with the dynamic observer (right);

FIG. 32 is a block diagram of an example of a system of the type shown in FIG. 1 with visual stimulus;

FIG. 33 shows a response kernal of a spike history model at 25 Hz, 50 Hz, 100 Hz and 200 Hz;

FIG. 34 shows a static nonlinearity using spike history model for 25 Hz, 50 Hz, 100 Hz and 200 Hz;

FIG. 35 shows the spike history model predictions with the response kernal vs. the spike history model predictions without the response kernal vs. recorded spike trains for 25 Hz, 50 Hz, 100 Hz and 200 Hz; and

FIG. 36 shows the auto-correlation function of the recorded spike train, the auto-correlation function of the spike history model with the response kernal, and the auto-correlation function of the spike history model without the response kernal for 200 Hz stimulation.

### DETAILED DESCRIPTION

Embodiments of the invention provide a stimulus generator arranged to determine an electrical stimulus to be applied to one or more neurons. In some embodiments, the stimulus generator is provided in a prosthetic apparatus such as a prosthetic device. In other embodiments, the invention is employed as a method in a prosthetic apparatus, for example by program code executed by a processor of a prosthetic device.

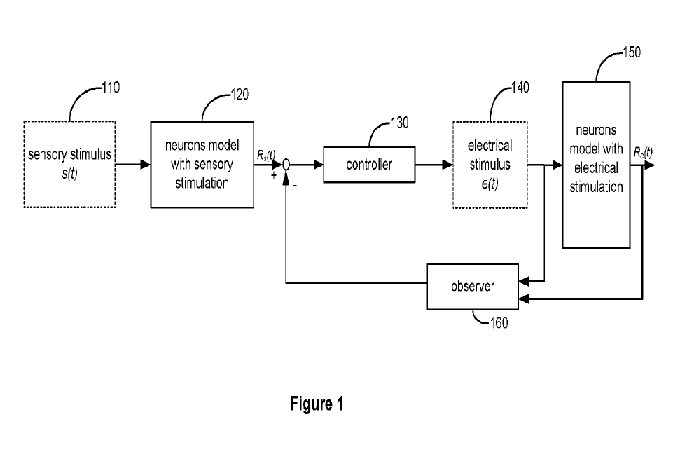

As shown in the block-diagram of FIG. 1, in one embodiment, a stimulus generator 100 employs a feedback system. FIG. 1 shows that a controller 130 can be designed to control based on a reference signal that is generated based on a modeled neuron response 120 to sensory stimulation 110, s(t), (and hence the modeled response 120 provides a reference signal generator). As can be seen in FIG. 1, the response 120 of a neuron to sensory stimulation, Rₛ, is used as a reference that is supplied to the controller 130 which generates an electrical stimulus e(t) to be applied to the neurons. The electrical stimulus e(t) is also supplied to a neuron modelling component 150 which determines a modelled response Rₑ of a neuron to the electrical stimulation 150 e(t) determined by the controller. The modelled response Rₑ is also fed back to the controller 130 via observer 160 to calculate the electrical stimulus 140 (and hence the calculation of the electrical stimulus in the next time period takes into account a modelled response of at least one prior period when determing the electrical stimulation e(t), to be applied. The observer 160 also takes into account the electrical stimulation that produced the response.

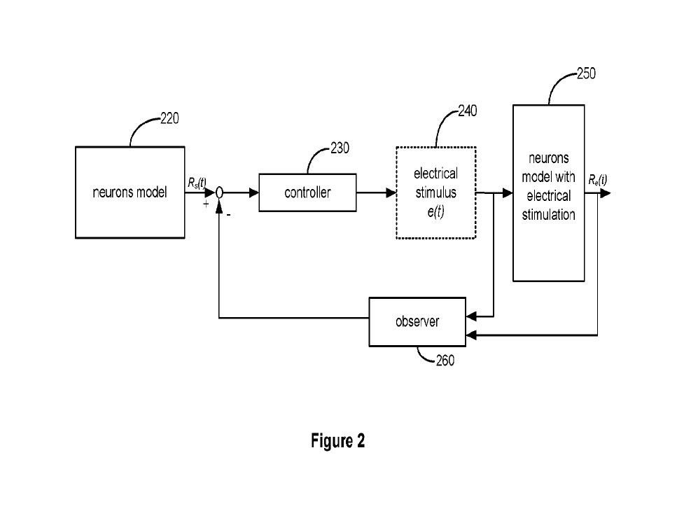

In other embodiments, a stimulus generator 200 is provided by a controller 230 attempting to achieve a target response based on a neuron model 220 that generates a reference signal corresponding to the expected behaviour of healthy neurons. As shown in FIG. 2, in such embodiments, there is no sensory stimulation. In a manner analogous to FIG. 1, a neuron modelling component 250 determines a response of a neuron to electrical stimulation, Rₑ, that is used by the observer 260 in order to provide a feedback signal to the controller 130.

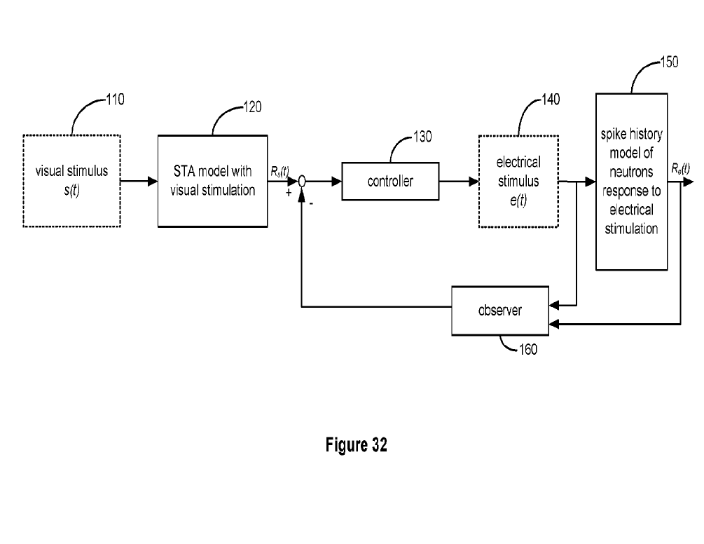

Employing a feedback system in a stimulus generator 100, 200, as shown in FIG. 1 or FIG. 32, provides means to adjust the parameters of a prosthetic device based on the neuron's response. An example of a stimulus generator 101 for a visual prosthetic device is given in FIG. 32, which shows that the specific form of stimulus is a visual stimulus 111, the reference signal generator 121 employs a spike-triggered average model with visual stimulation and a spike history model of neuron response to electrical stimulation 151 is used in generating the applied stimulus.

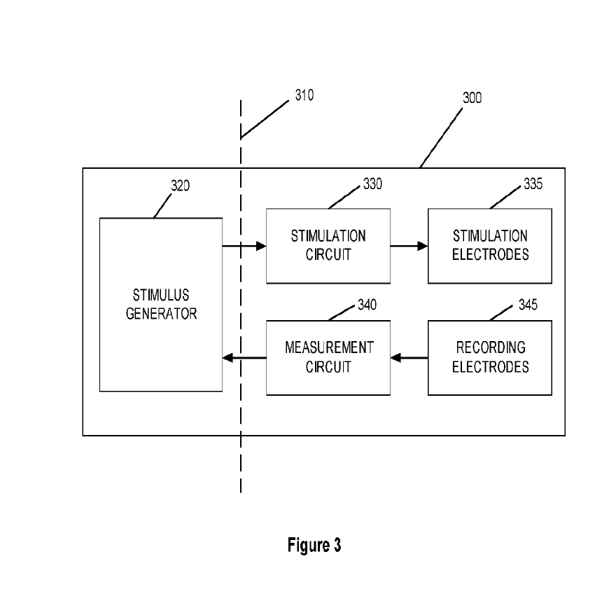

An embodiment of a stimulus generator 320 in a neuroprosthetic apparatus 300 for visual stimulus is shown in FIG. 3. As is known in the art, some components of the prosthetic apparatus 300 are designed for implantation and some components are located externally. One example of the division between internal and external components is indicated by dotted line 310 in FIG. 3 but persons skilled in the art will understand that the delineation between external and internal components will vary depending on implementation. In the example of FIG. 3, the stimulus generator 320 is an external component while stimulation circuit 330, stimulation electrodes 335, recording electrodes 345, and measurement circuit 340 are each implanted. It will be appreciated that the number and nature of the components that are for implantation will vary depending on implementation. Further, the stimulus generator 320 may be formed of a number of sub-components, some of which are implanted and some of which are not. Further, the stimulation circuit 330 may form part of the stimulus generator 320 in some embodiments. Communication and power supply between internal and external components can be achieved using wires extending through tissue or via electromagnetic excitation. While separate stimulation 335 and recording electrodes 345 are shown in FIG. 3, in some embodiments, the same electrodes may both stimulate and record (for example in different parts of a stimulation cycle). Further, it will be appreciated that depending on the embodiment, the measured response may be of a specific neuron or a population of neurons. The neuron response may directly correspond to the neuron now being stimulated or may be sufficiently related to be a proxy for the prior response of the one or more neurons now being stimulated. For example, the measured response may correspond to a larger population of neurons than those that will be stimulated or vice versa.

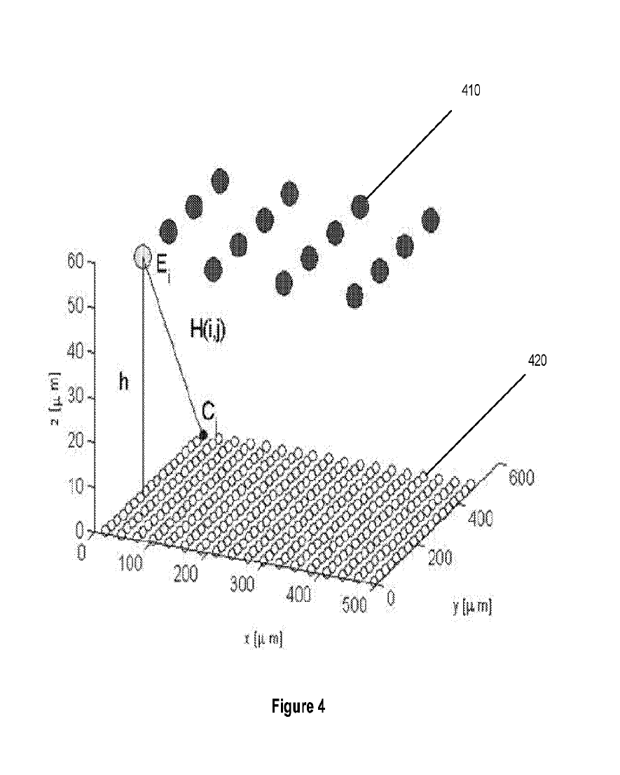

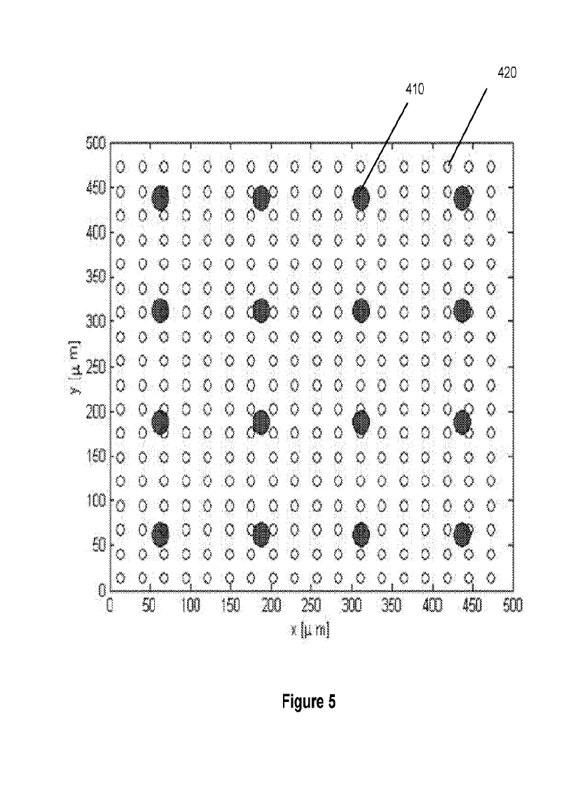

A schematic diagram of an example of an electrode array for a prototype visual prosthesis is shown in FIGS. 4 and 5. FIG. 4 is a perspective view of a 4×4 point electrode array 410 placed at height h=60 μm above tissue 420 where the tissue area is 0.5 mm². The distance between electrodes is 125 μm. Cell density is 1250 cells/mm². The distance from an electrode Eᵢ to a cell Cⱼ is represented by the matrix H(i,j). A plan view of the array of electrodes 410 and tissue 420 (the z-axis is collapsed) is given in FIG. 5.

Cell density is assumed to be uniform. In healthy human retina, an average density of retinal ganglion cells is 2395/mm. (See Harman A., Abrahams B., Moore S., Hoskins R. Neuronal density in the human retinal ganglion cell layer from 16-77 years. The Anat. Record, 260: 124-131, 2000.) While the cell density depends on eccentricity, and 2395 cells/mm² is for peripheral retina, and centrally there are more than 10⁵ cells/mm², the lower density number is employed to simplify the model. According to Medeiros N. F., Curcio. C. A. Preservation of ganglion cell layer neurons in age-related macular degeneration. Invest. Ophthalmol. Vis. Sci., 42: 795-803, 2001, 53% of Retinal Ganglion Cells (RGCs) survive in a retina of a person with Age-related Macular Degeneration (AMD). Therefore, the embodiment assumes that density of RGCs in degenerative retina is 1269 cells/mm² (317 cells in 0.5 mm² tissue). In order to have a rectangular grid of cells, this value is rounded to 324 corresponding to an 18×18 array of electrodes. In the description that follows, n is a number of neurons and m is the number of electrodes, as they may vary. In other embodiments, cell density may vary.

The array is assumed to be h=60 μm from the tissue. A distance H_{i,j} from every electrode (i=1, 16) to every cell (j=1, 324) is given in the matrix H. It is calculated as follows:

H_{i,j}=(h²+|xⱼ−xᵢ|²+|yⱼ−yᵢ|²)^{1/2}, (1)

where (xᵢ, yᵢ, h) are the coordinates of the electrode i and (xⱼ, yⱼ, 0) are the coordinates of the cell j. The distance H_{i,j} from the highlighted electrode Eᵢ to the cell Cⱼ is shown in FIG. 4.

The influence of individual electrodes i onto individual cells j is calculated according to:

$${\hat{V}}_{j} ( ω ) = \frac{b \hat{e}}{8 Π {H}_{i , j}^{3} {C}_{m} ( {τ}_{PS}^{- 1} + jω )} . \qquad ( 2 )$$

The influence of all electrodes onto the cell j is calculated as follows:

$${\hat{V}}_{j} ( ω ) = \frac{b}{8 Π {C}_{m} ( {τ}_{PS}^{1} + jω )} {\sum }_{l = 1}^{m} \frac{\hat{e}}{{H}_{i , j}^{3}} . \qquad ( 3 )$$

The influence of the electrode is scaled by the cubed distance between the electrode and the cell. \hat{V} is an intracellular voltage potential, êᵢ is a current applied at an electrode i. Hats above variables describe Fourier transform in time. Cₘ=10⁻¹⁴ Fμm², τ_{PS}=1.1 ms, b=0.5 μm. Bold j is an imaginary unit. Not bold j defines the subindex. Cₘ is the membrane capacitance constant, τ_{PS} is the membrane time constant, b is the outer radius surrounding the intracellular part of the cell.

The membrane voltage of equation (3) is then converted into neural spiking rate using a firing-rate model:

R=f(Vₘ) (5)

where Vₘ is the intracellular membrane potential, that can be found using the inverse Fourier transform of (3) above.

The output measurements of a neural population spike rates measured at the electrode i are calculated as follows:

$${R}_{NeuralPopulation} ( k ) = \frac{1}{4 Π} {\sum }_{l = 1}^{n} \frac{{R}_{l} ( k )}{{H}_{l , j}^{3}} , \qquad ( 4 )$$

where t is time.

In one example of how the influence of an electrode current on a neuron can be estimated, a neural spike rate can be estimated by employing a spike-triggered average (STA) method described in: Chichilnisky E. J. A simple white noise analysis of neuronal light responses. J. Comput. Neural Syst., 2001, vol. 12, pp. 199-213; Dayan P., Abbott L. F. Neural encoding I: firing rate and spike statistics. MIT Press, Cambridge, 2001; and Klein D., Depireux D., Simon J., Shamma S. Spectro-temporal methods in primary auditory cortex. Publications of Center for Auditory and Acoustic Research.

The STA is the average stimulus preceding a neuron's spike. The STA method characterizes the response properties of a neuron using the spikes emitted in response to a time-varying stimulus. This method describes spatial, temporal and spectral response properties of spiking neurons. The method is relatively robust to fluctuations in response, avoids adaptation to strong and prolonged stimuli and is well-suited to simultaneous measurements from multiple neurons as described in Chichilnisky E. J. A simple white noise analysis of neuronal light responses. J. Comput. Neural Syst., 2001, vol. 12, pp. 199-213.

The STA method is based on Gaussian white noise stimulation to obtain parameters of the STA and nonlinearity of neural response. For white-noise visual stimulation, STA is a vector with values of an intensity of light from the whole spectrum. To compute the STA, the stimulus in the time window preceding each spike is extracted, and the resulting (spike-triggered) stimuli are averaged at each point in time, an image of an intensity drawn from a Gaussian distribution is presented. Then, the stimulation vector is a collection of intensities, s(t), that were presented in a time window prior to prediction time. For example, if a time window corresponds to five stimulation points, to predict response of a neuron at the time t₁₁, a vector (s(t₆), s(t₇), s(t₈), s(t₉), s(t₁₀) is used.

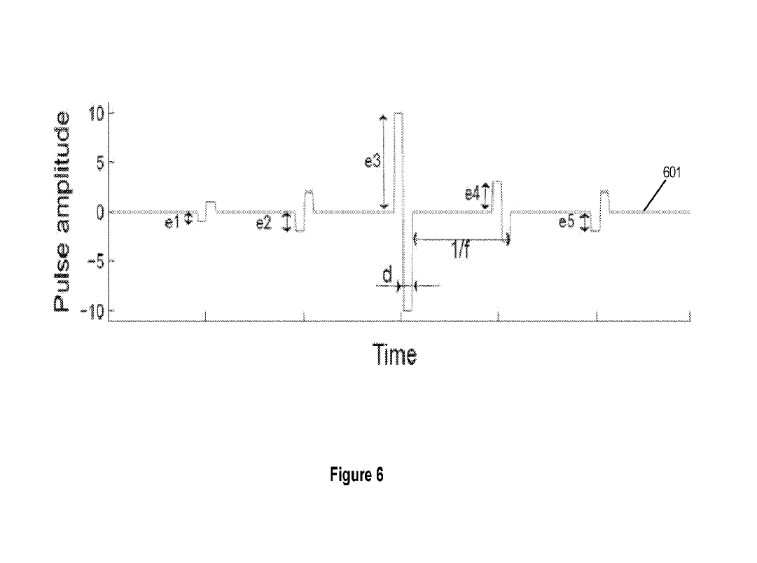

For Gaussian white noise electrical stimulation, an amplitude of a bi-phasic pulse is drawn from a Gaussian distribution with zero mean and variance of one. An example of a bi-phasic pulse train is given in FIG. 6, where e₁, e₂, e₃, e₄, e₅ are the amplitudes of consecutive pulses, negative amplitude corresponds to an anodic pulse first, f is a frequency of a pulse train, and d is a pulse width. A vector \vec{e}=e₁, e₂, e₃, e₄, e₅ represents a pulse train 601 used to estimate a spiking rate of a neuron. It is assumed that interphase gap is zero (can be non-zero in general), d is the same for anodic and cathodic pulses, d<<1/f.

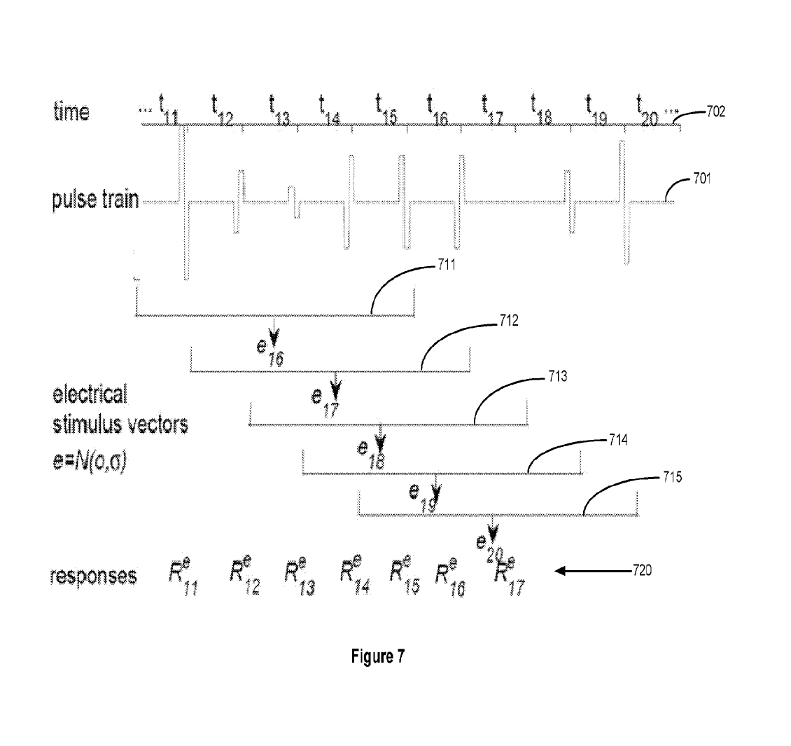

An example of Gaussian white noise pulse stimulation is given in FIG. 7 which is illustrates the relative timing of the pulse train 701 stimulus vectors 711-715 and responses 720. Similar to the above, a pulse train 701 is applied by pulses at defined time periods 702. This results in a series of electrical stimulus vectors e₁₆, e₁₇ . . . e₁₉ 711-715. To estimate the response R₁₇ of a neuron at a time t₁₇, a stimulus \vec{e}₁₇ is used.

The time-varying firing rate of a neuron is estimated as in the following:

R^{s}(t)=G^{s}(\vec{g}ₛ·\vec{s}), (6)

R^{e}(t)=G^{e}(\vec{g}ₑ·\vec{e}), (7)

where R^{s}, R^{e} are the firing rates of a neuron in response to sensory and electrical stimulations, respectively, G^{s}, G^{e} are the nonlinearities for sensory and electrical stimulations (generator functions), \vec{g}ₛ, \vec{g}ₑ are the STAs for sensory and electrical stimulations, \vec{s}, \vec{e} are the sensory and electrical stimuli, and • is a dot product.

When firing rate of a neuron is plotted as a function of a convolution of the STA and the applied stimulus for a particular experiment, it is possible to obtain nonlinearity G^{s} (and G^{e}) and predict R^{s}(t) (and R^{e}(t)) for any stimulus. A spike train is then generated from a Poisson process.

While nonlinearities for both sensory and electrical stimuli have saturation levels, G^{s} can be fitted by a sigmoidal function, while G^{e} is a two-sided function because a cathodic first or anodic first pulse may both cause spikes.

In embodiments where it is intended to stimulate neurons in response to sensory stimulation, the response of neurons to sensory stimulation, R^{s}, is used as a reference signal. The reference is application dependent. In this example, the reference is the response to a visual stimulus according to the neuron model Response of neurons to electrical stimulation, R^{e}, is used for controller design.

In another embodiment, the inventors propose an alternative method for estimating the neural response. In this spike history model, the spike rate at time t depends not only on the stimulus in a short time window preceding the time t, but also on the response of a neuron in a time window preceding the time t.

To incorporate spike history memory into the model, the time-varying firing rate of a neuron, as set out in equation (7), is modified as follows:

R(t)=G(ae[t−1]+|e[t−1]|+h·\vec{r}_{[t-t}_{₁}_{, t-1]}), (8)

where \vec{h} is a kernel for the recent neural response. In other words, response of a neuron at time t depends on a spike history during [t−t₁, t−1], where t₁−1 is the dimension of \vec{r}. Note, subindexes s,e in R,G for the light and electrical stimulations are dropped. Unless overwise stated, the derivations are true for both light and electrical stimulations. The parameter a is optimised as described below.

To find G and h, it is assumed that G can be approximated by a hyperbolic tangent:

$$G ( x ) = \frac{1 + tanh [ c ( x - d ) ]}{2}$$

and h is approximated by a double exponential function:

h(t)=A₁e^{-t/τ}^{₁}+A₂e^{-t/τ}^{₂}.

Then the probability of a spike at each time step, t, is calculated as follows:

$${P}_{k} = \{ \begin{matrix}G if a spike was recorded at k \\ 1 - G if no spike was recorded at k\end{matrix} \qquad ( 9 )$$

The log-likelihood of observing the same spike train as experimentally recorded, is calculated as follows:

$$L = {\sum }_{k = 1}^{{T}_{end}} ln ( {P}_{k} ) ,$$

where T_{end} is a number of sample points in an experiment.

Parameters a, c, d, A₁, τ₁, A₂, τ₂ are then optimized to maximize the log-likelihood of L.

It will be appreciated that the above models of neural response are examples only and other models of neural dynamics may be used.

Below is one embodiment of a controller design. Other controller designs may be used. To assist in the design of the controller, the nonlinearity of equation (5) is initially disregarded and the system is modeled using equations (3) and (4) only. Subsequently, a nonlinear block is added that takes into account the nonlinearity.

Without loss of generality, the following substitutions can be done for each electrode in equation (3):

$${\hat{V}}_{j} ( ω ) = {x}_{j} ( x ) , {\hat{e}}_{i} = {u}_{i} , \frac{b}{8 π {C}_{m}} {\sum }_{l = 1}^{n} \frac{1}{{H}_{1 , j}^{3}} = {\hat{B}}_{j} , j = 1 , … , n ;$$

$$i = 1 , … , m .$$

For an array of electrodes, this results in the following matrix form:

$$x = [ \begin{matrix}{\hat{V}}_{1} \\ ⋮ \\ {\hat{V}}_{n}\end{matrix} ] , u = [ \begin{matrix}{\hat{e}}_{1} \\ ⋮ \\ {\hat{e}}_{n}\end{matrix} ] , {\hat{B}}_{j} = \frac{b}{8 π {C}_{m}} [ \begin{matrix}\frac{1}{{H}_{1 , j}^{3}} & … & \frac{1}{{H}_{m , j}^{3}}\end{matrix} ] .$$

Then equation (3) can be written in the following state-space representation:

$$\dot{x} = Ax ( t ) + Bu ( t ) , \\ where$$

$$A = [ \begin{matrix}τ & 0 & 0 \\ 0 & ⋱ & 0 \\ 0 & 0 & τ\end{matrix} ] , B = [ \begin{matrix}{\hat{B}}_{1} \\ ⋮ \\ {\hat{B}}_{n}\end{matrix} ] . , \\ τ = 1.1 ms .$$

The output measurements in state-space representation are defined as follows:

$$y ( t ) = Cx ( t ) , C = [ \begin{matrix}\frac{1}{{H}_{1 , 1}^{3}} & … & \frac{1}{{H}_{1 , m}^{3}} \\ ⋮ & ⋱ & ⋮ \\ \frac{1}{{H}_{n , 1}^{3}} & … & \frac{1}{{H}_{n , m}^{3}}\end{matrix} ] .$$

Then the full state-space representation of the system is as follows:

$$\{ \begin{matrix}\dot{x} = Ax ( t ) + Bu ( t ) & x ( t ) ∈ {ℛ}^{n} , u ( t ) ∈ {ℛ}^{m} . \\ y ( t ) = Cx ( t ) & y ( t ) ∈ {ℛ}^{m}\end{matrix} . \qquad ( 10 )$$

In the above, vector x represents a collection of n neurons, the elements of vector y are the measurements at each of m electrodes and vector u represents the stimulation amplitude at each electrode. The system of equations (10) is detectable but not completely observable. In order to draw conclusions about the system's detectable and observable subspace states, a Kalman decomposition of equation (10) is used that converts equation (10) into the controllable/observable (c,o), the not controllable/observable ( c̅,o), the controllable/not observable (c,ō), and the not controllable/not observable ( c̅,ō) parts of the system.

Using Kalman decomposition, we transform (A,B,C,D) into (Ā, B̅, C̅, D̅) using the following formula:

Ā=T⁻¹AT, B̅=T⁻¹B, C̅=CT, D̅=D; z(t)=T⁻¹x(t), (11)

where T is a suitable n×n invertible matrix and z(t) is the new state of the system. Using (11) we have Ā, B̅ and C̅ in the following forms:

$$\overline{A} = [ \begin{matrix}{\overline{A}}_{c , o} & 0 & {\overline{A}}_{1 , 3} & 0 \\ {\overline{A}}_{2 , 1} & {\overline{A}}_{c , \overline{o}} & {\overline{A}}_{2 , 3} & {\overline{A}}_{2 , 4} \\ 0 & 0 & {\overline{A}}_{\overline{c} , o} & 0 \\ 0 & 0 & {\overline{A}}_{4 , 3} & {\overline{A}}_{\overline{c} , \overline{o}}\end{matrix} ] , \overline{B} = [ \begin{matrix}{\overline{B}}_{c , o} \\ {\overline{B}}_{c , \overline{o}} \\ 0 \\ 0\end{matrix} ] , \\ \overline{C} = [ \begin{matrix}{\overline{C}}_{c , o} & 0 & {\overline{C}}_{\overline{c} , o} & 0\end{matrix} ] .$$

Then, the full state-space representation of the transformed system is:

$$\{ \begin{matrix}\dot{z} ( t ) = \overline{A} z ( t ) + \overline{B} u ( t ) \\ y = \overline{C} z ( t )\end{matrix} , \\ where \\ z ( t ) = [ \begin{matrix}{z}_{r} \\ {z}_{c , \overline{o}} \\ {z}_{\overline{c} , o} \\ {z}_{\overline{c} , \overline{o}}\end{matrix} ] . \qquad ( 12 )$$

The upper right and lower left blocks of Ā (that correspond to the not controllable/observable, and not observable/controllable parts of the system) lower block of B̅ and right block of C̅, had values close to zero:

$$\overline{A} = [ \begin{matrix}{\overline{A}}_{c , o} ≅ 0 \\ ≅ 0 {\overline{A}}_{\overline{c} , \overline{o}}\end{matrix} ] , \overline{B} = [ \begin{matrix}{\overline{B}}_{c , o} \\ ≅ 0\end{matrix} ] , \overline{C} = [ \begin{matrix}{\overline{C}}_{c , o} & ≅ 0\end{matrix} ] \qquad ( 13 )$$

In the following description only the controllable and observable part of the system is considered for the feedback controller gain and observer designs, i.e.:

$$\{ \begin{matrix}{\dot{z}}_{r} ( rt ) = {\overline{A}}_{r} {z}_{r} ( t ) + {\overline{B}}_{r} u ( t ) \\ y = {\overline{C}}_{r} {z}_{r} ( t )\end{matrix} , \\ where \\ {\overline{A}}_{r} = {\overline{A}}_{c , o} , {\overline{B}}_{x} = {\overline{B}}_{c , o} , {\overline{C}}_{x} = {\overline{C}}_{c , o} \qquad ( 14 )$$

and zₓ correspond to the controllable and observable part of the state in the Kalman decomposition form.

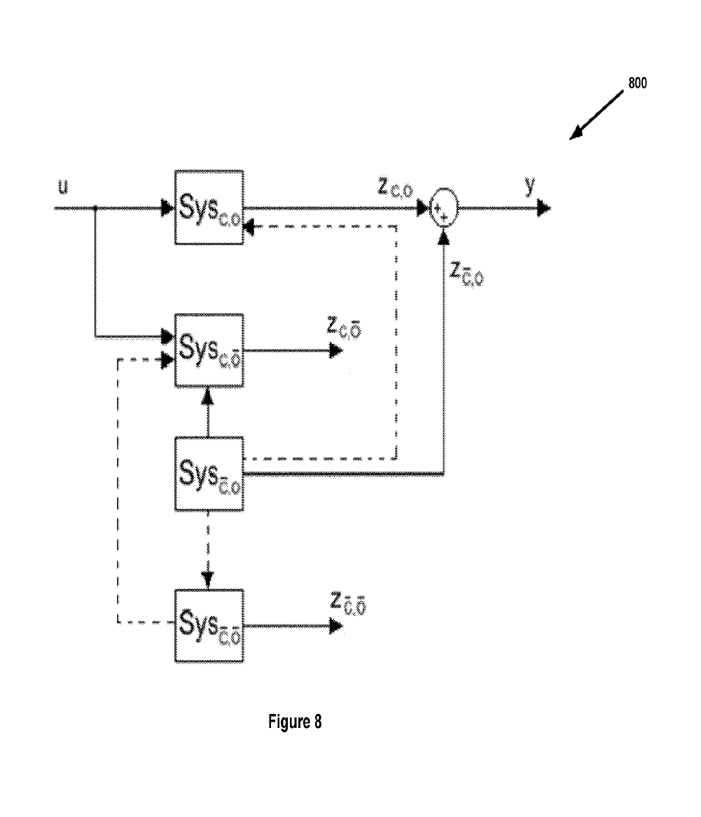

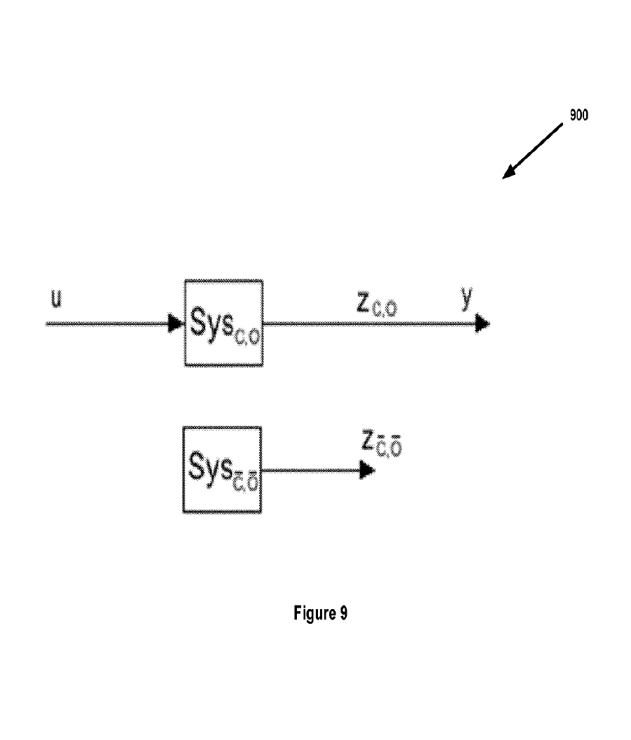

Kalman decomposition 800 of a general system of linear equations is shown in FIG. 8. Kalman decomposition 900 of the system (10) is shown in FIG. 9 due to the not controllable/observable, and controllable/not observable parts of the system absent, in accordance with equation (13).

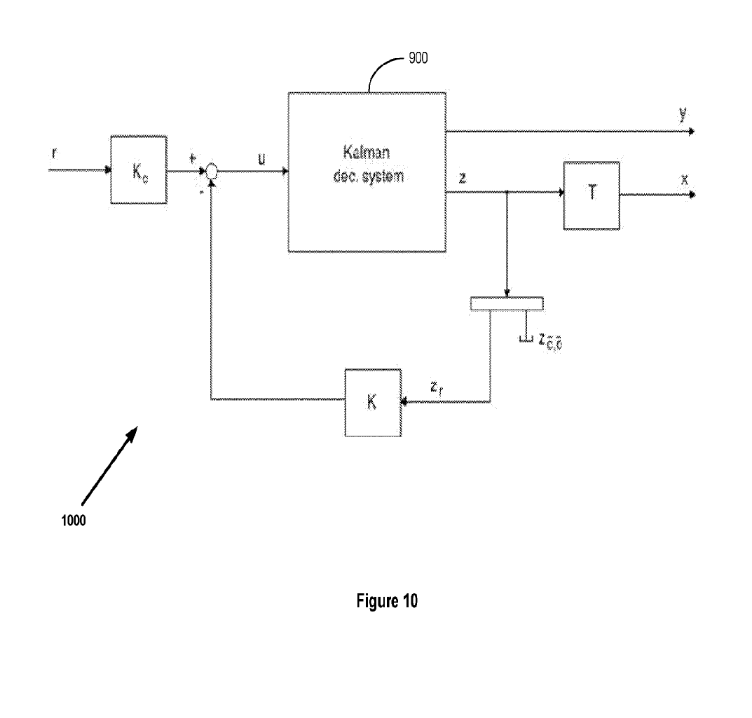

To control the system 1000 in one embodiment illustrated in FIG. 10, a linear quadratic regulator (LQR) is implemented. The LQR provides a feedback gain, K, in

u(t)=−Kzᵣ(t).

The technique is based on minimizing the quadratic cost function J in

J= [symbol] (x^{T}Qx+u^{T}Ru)dt,

R>0 and Q≧0 are found by solving the continuous time Riccati equation:

A^{T}P+PA−PBR⁻¹B^{T}P=−Q.

The following R and Q, that satisfy conditions above, were employed:

$$R = [ \begin{matrix}{r}_{1} & 0 & 0 \\ 0 & ⋱ & 0 \\ 0 & 0 & {r}_{m}\end{matrix} ] , Q = [ \begin{matrix}{q}_{1} & 0 & 0 \\ 0 & ⋱ & 0 \\ 0 & 0 & {q}_{m}\end{matrix} ] ,$$

where rᵢ=10, qᵢ=10⁻⁸, i=1 . . . m. In order to minimize a metric of the tracking error, a feedforward controller with gain K_{c} was included in the system 1000 of FIG. 10:

$${K}_{c} = [ \begin{matrix}{k}_{{c}_{1}} & 0 & 0 \\ 0 & ⋱ & 0 \\ 0 & 0 & {k}_{{c}_{m}}\end{matrix} ] .$$

The resulting controller has the following form:

u=K_{c}r−Kzᵣ,

where r is a reference.

In the original system of equations (10) there are m inputs, which correspond to the applied current amplitude at each of m electrodes. However, when an image or a video is used as a reference, a number corresponding to each pixel of an image (video frame) is given, this number corresponds to n neurons, n>m. The transformation of the old reference into the new reference signal is based on the following:

r=Crₓ [symbol] r≅x,

where rₓ is a new reference signal and r is an old reference, since

$$\begin{matrix}y ≅ r \\ y = Cx\end{matrix} -> r ≅ Cx .$$

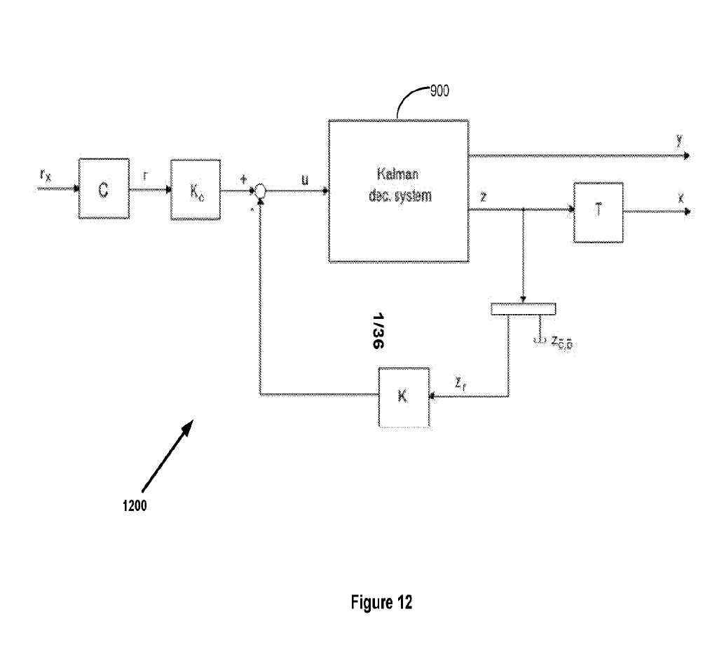

A diagram of an embodiment of the feedback system 1200 with the new reference signal is shown in FIG. 12.

In a further embodiment, the reference signal is calculated according to equation (6) depending on the light intensity at each point in space and is updated dynamically on-line. In some embodiments, the reference signal will be different for each electrode or may represent other than light intensity.

In order to test the embodiments by simulations, two types of references were used.

Firstly, the reference was taken as an average of in-vitro recordings of a spike rate of eight individual retinal ganglion cells of a primate in response to natural optical stimuli in laboratory environment were employed as described in H., Ruttiger L, Sun H., Lee B. B. Processing on natural temporal stimuli by macaque retinal ganglion cells. J. Neurosci, 15: 9945-9960, 2002.

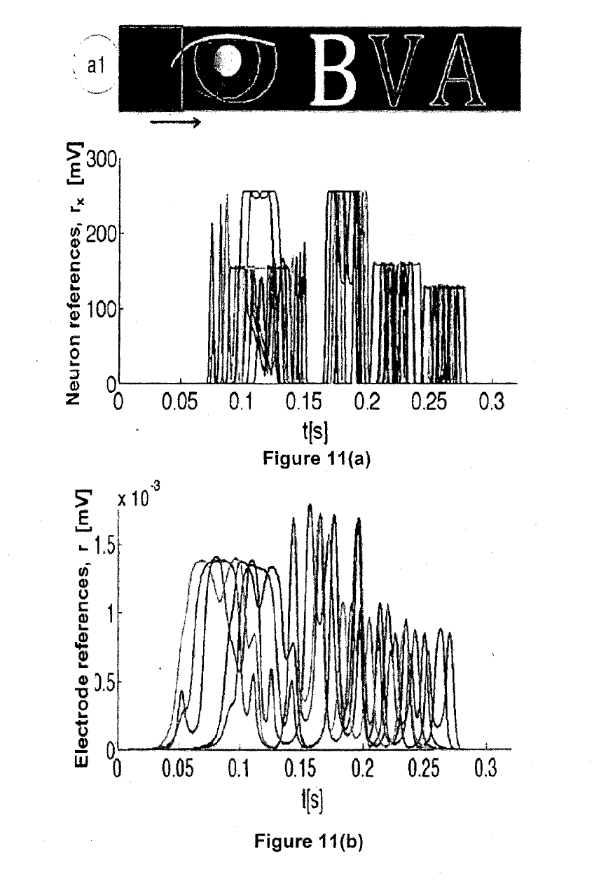

Secondly, the reference was taken as a spike rate that was proportional to an average light intensity around each electrode in a movie frame. FIG. 11 shows this reference for neurons (top) and for electrodes (bottom) when the function f in the equation (5) is identity. For these simulations, the image scrolls from right to left in 0.3 s. The subplot (a1) shows a reference image. The analyzed area is represented by a red square in subplot (a1). While simulations were done with 144 electrodes, only a subset of the references of the system is illustrated for clarity. Different traces correspond to different neurons/electrodes.

According to equation (3), all electrodes have an effect on cell j. In reality, only electrodes in close enough proximity to the cell j may influence its membrane potential. This more realistic physiological condition was imposed by assuming that only neurons within a radius a around the electrode i can be affected by this electrode. In other words,

if (He_{i,j}>a) [symbol] C_{i,j}≡0. (15)

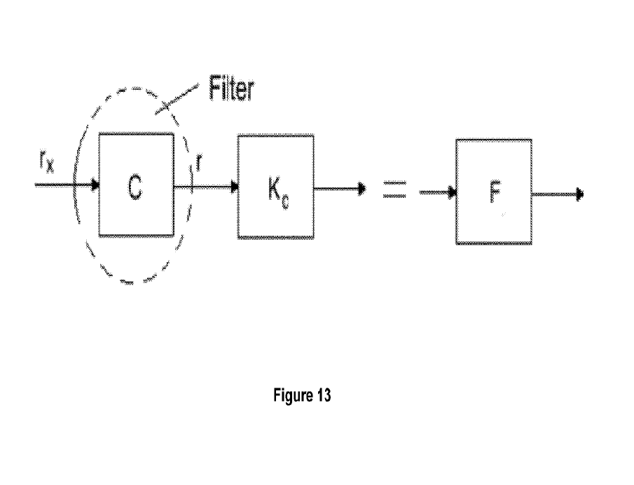

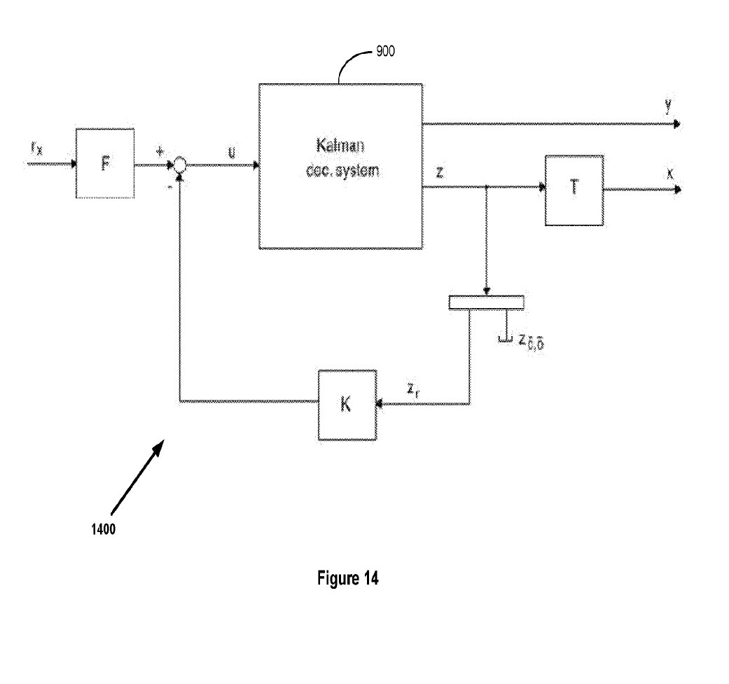

Another way this can be thought is if H_{i,j}>a) in (3) [symbol] Iₑ≡0. This allows a further embodiment of the feedback system that employs a filter as per equation (15) and as illustrated schematically in FIG. 13. Such an embodiment is shown in FIG. 14 from which it can be observed that Filter (F) replaced components C and K_{c}.

Observer Design

In order to implement a static observer, only the controllable and observable parts of the system (10) are considered, i.e. the equations (14). The output of the controllable and observable part of the system (14) has the following form:

ti y= C̅ᵣzᵣ(t)

When the matrix C̅ᵣ is a square matrix, it is possible to invert it. When the matrix C̅ᵣ is not a square matrix (a number of electrodes is not equal to the number of neurons), a pseudo-inverse is used. Then the following equation is used for the static observer measurements:

zᵣ(t)= C̅ᵣ⁻¹y(t)

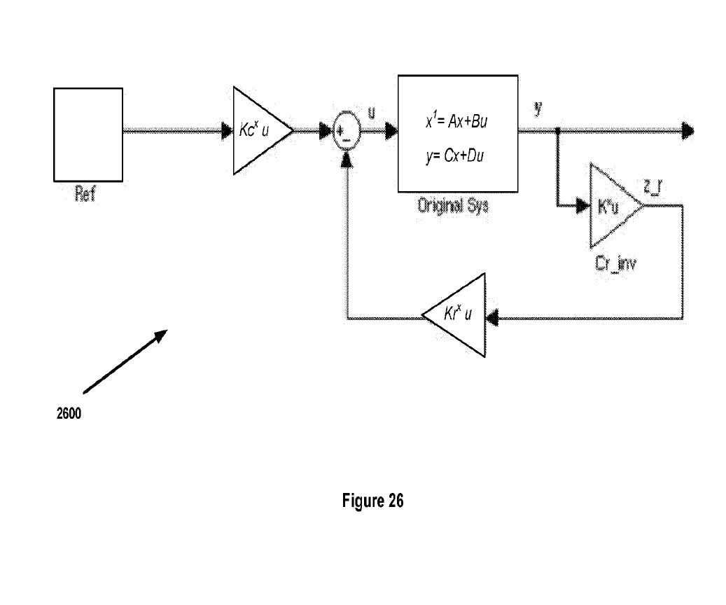

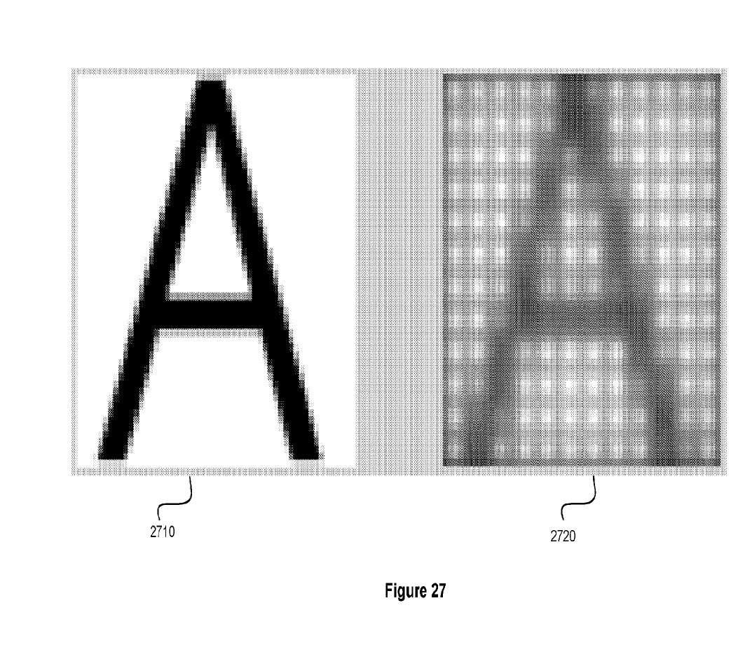

A system 2600 with a static observer is shown in FIG. 26. FIG. 27 shows the reference image 2710 and the output of the system with the static observer using 12 electrodes 2720.

In order to implement a dynamic observer, only the controllable and observable parts of the system (10) are considered, i.e. the equations (14). The matrixes Āᵣ, B̅ᵣ and C̅ᵣ are used in the Luenberger observer equations as following:

$$\{ \begin{matrix}{{z}^{\dot{\hat{}}}}_{r} ( t ) = {\overline{A}}_{r} {\hat{z}}_{r} ( t ) + {\overline{B}}_{r} u ( t ) + L ( y - \hat{y} ) \\ \hat{y} = {\overline{C}}_{r} {\hat{z}}_{r} ( t )\end{matrix}$$

The observer gain L is chosen such that the observer error converges to zero asymptotically. The observer error satisfies the following equation:

ė(t)=Āᵣ−L C̅ᵣ)e(t)

The eigenvalues of the matrix (Āᵣ−L C̅ᵣ) can be made arbitrarily by appropriate choice of the observer gain L because the pair [Āᵣ, C̅ᵣ] is observable.

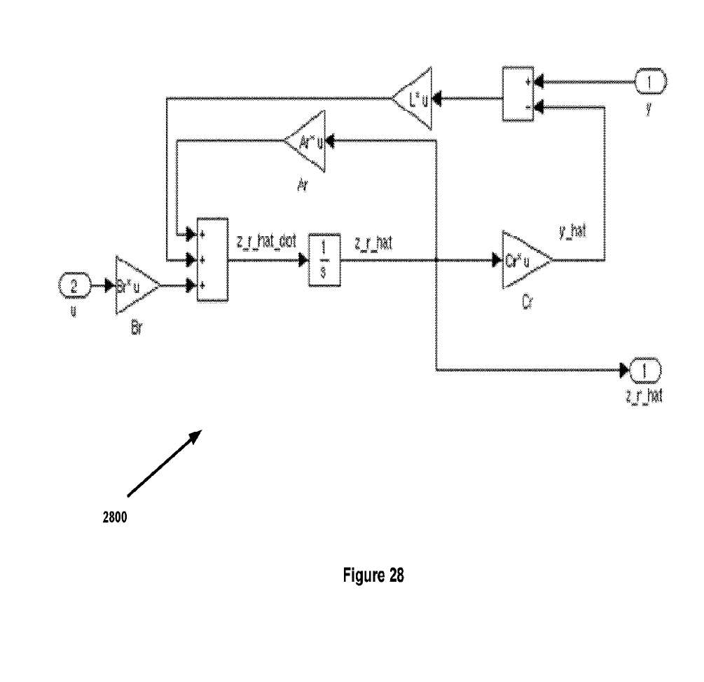

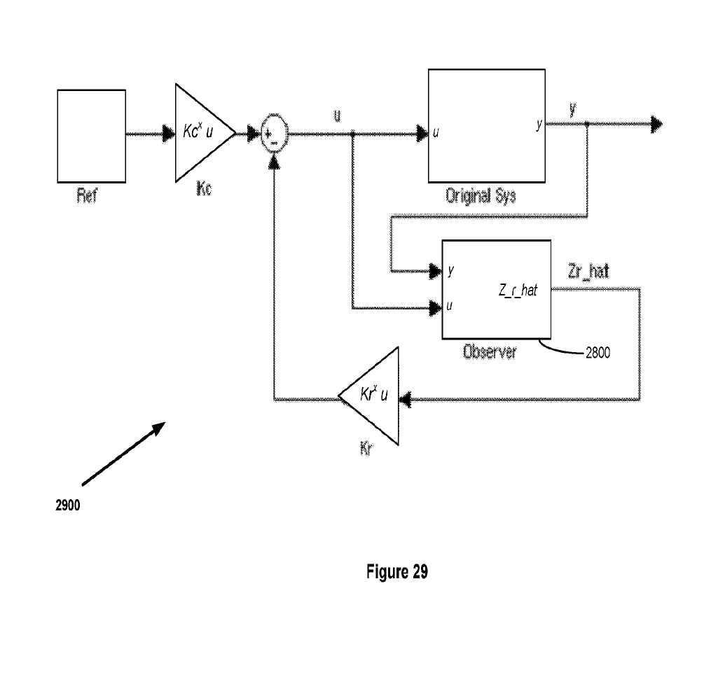

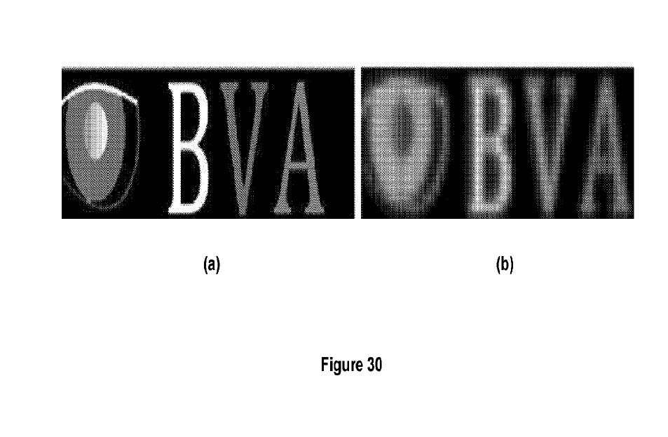

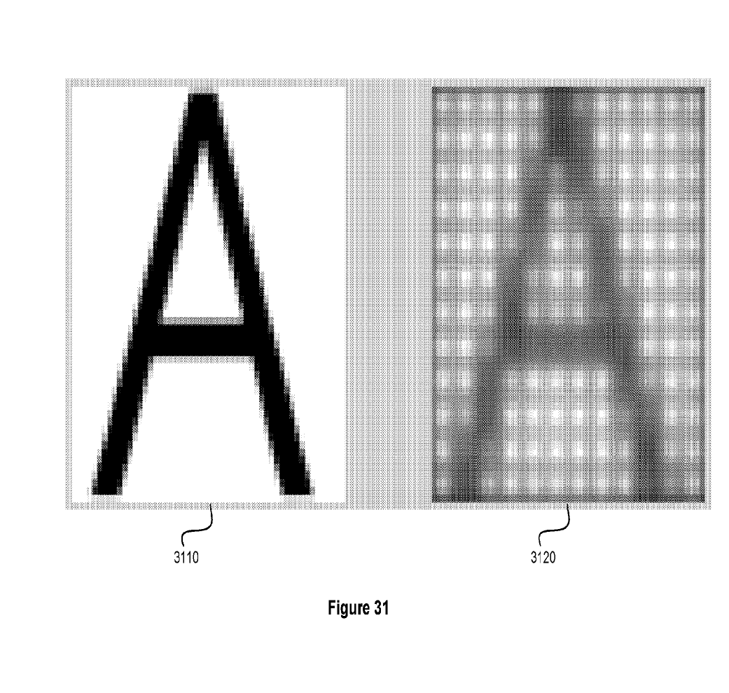

A diagram of the dynamic observer 2800 is given in FIG. 28 and the diagram of the feedback system 2900 with the dynamic observer is given in FIG. 29. FIG. 30 shows the reference image (left) and the state of the system with a static observer (right). FIG. 31 shows the reference image 3110 and the output 3120 of the system with the dynamic observer using 12 electrodes.

It will be appreciated from the above that, while many stimulation strategy algorithms in a bionic device have been shown being successful using feedforward techniques, the outcome differs from patient to patient. The feedback stimulation technique described above can address this variability by enabling tailoring stimulation strategy according to the measured response in a neuroprosthetic apparatus.

Additional benefits of using feedback in neuroprosthetic stimulation include:
i. More targeted stimulation, so that only the required amount of electrical stimulation is delivered. This reduces neural habituation, which can lead to loss of performance over time.
ii. The use of feedback reduces the power consumption, since a stimulator is only activated when required.
iii. Reducing power consumption is important from the patient's perspective since the device requires less batteries and/or longer time between recharges.
iv. Well-designed feedback has less potential for damage to stimulated tissue.

While the embodiments described above relate to apparatus such as visual prosthetic devices, they can similarly be applied to auditory implants which are designed to provide stimulation in response to auditory input in an analogous way to the above embodiments. The techniques described above also have potential wider application to provide improvements in for example seizure suppression, faster modulation of neural synchrony in patients with Parkinson's disease. For example, Parkinsonian resting tremor is caused by a population of pacemaker-like neurons firing synchronously. In healthy subjects, this population of neurons fire in an uncorrelated and non-periodic way. At present, a strategy to stop neuronal synchronization (and, therefore, tremor) is to apply electrical stimulation (usually at 130 Hz) to a pathological neural population continuously. This continuous stimulation may lead to some undesirable effects, such as neural adaptation and potential for damage of stimulated neural tissue.

It is envisaged that it will be possible to employ a feedback stimulation strategy for patients with Parkinson's disease as illustrated in FIG. 2. An output of a model 220 of healthy neural population is used as a reference to a controller 230. Electrical stimulation 240 is adjusted dynamically based on the recent neural response, Rₑ(t), to the stimulation as observed by observer 260. Electrical stimulation is optimized in a way such that neural response, Rₑ(t), closely approximates the reference, i.e. the output of the model of a healthy neural population.

Such strategies have many advantages compared to the currently used continuous stimulation, including (i)-(iv) above and the fact that it allows the strategy to mimic healthy neural dynamics.

Similarly, epilepsy is a neurological disorder where seizures occur randomly, normally caused by over-excitation of populations of neurons. While drugs and surgery can be used to control epileptic seizures, 25% of people suffering from epilepsy cannot be treated sufficiently by currently available therapies. A part of this population is suitable for treatment of seizures by electrical stimulation. Currently, neural response is used only for seizure detection or prediction and not for optimization of stimulation parameters. The amplitude or frequency of stimulation is often drawn from a white noise or Poisson distribution and is not based on dynamics of a healthy neural population.

Similar to the stimulation strategy for patients with Parkinson's disease, we propose to use an output of a model of healthy neural population as a reference to a controller. Electrical stimulation is adjusted dynamically based on the recent neural response, Rₑ(t), to the stimulation. Electrical stimulation is optimized in a way so that neural response, Rₑ(t), closely approximates the reference, i.e. the output of the model of a healthy neural population.

Experimental Data

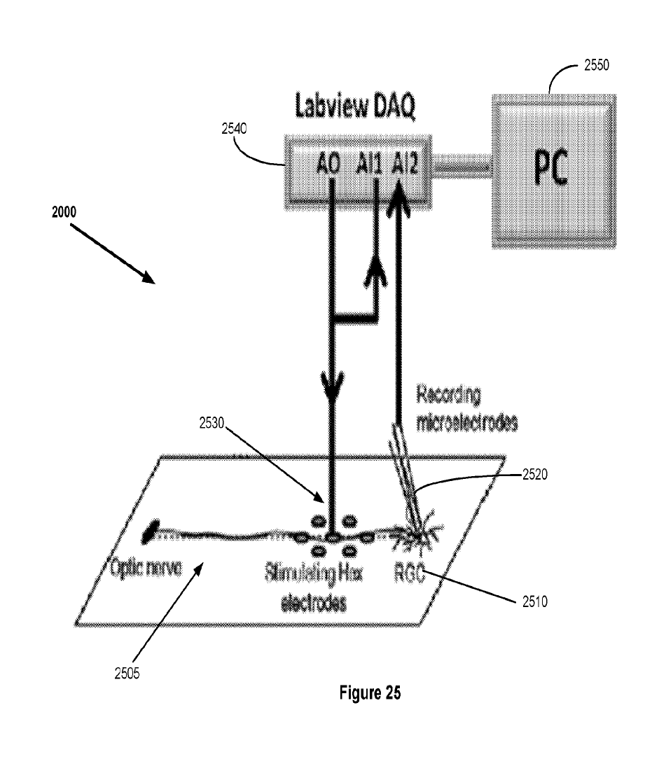

To validate parameters in the models (5), (7), (8), the following exemplary experimental set up 2500 (shown schematically in FIG. 25) was employed. This is one embodiment. Various experimental protocols with single or multiple stimulation and recording electrodes can be used.

Tissue preparation: a piece of inferior retina 2505 obtained from a NZ white rabbit eye was placed flat, ganglion cell 2510 side up, in a perfusion chamber.

Recording electrodes: a Tungsten microelectrode pair 2520 was used for differential extracellular recording.

Stimulating electrodes: Seven platinum disk electrodes 2530 arranged hexagonally were used for epiretinal electrical stimulation. Electrode diameter and centre-to-centre spacing were 125 μm and 325 μm, respectively.

Stimulation and recording protocol: Recording microelectrodes were lowered onto the retinal surface to record action potentials from a ganglion cells. Stimulating electrodes were placed on the retinal surface between the recording electrodes and the optic nerve, along the inferred axonal path.

A PC 2550 running LabVIEW software available from National Instruments of Austin, Tex., USA was used to control electrical stimulation and data acquisition.

Electrical stimulation: A train of 5000 biphasic current pulses with 100 us phase duration was used for stimulation. Stimulus frequencies were 25, 50, 100, 200,500 and 1000 Hz. Square bipolar voltage pulses were delivered by LabVIEW DAQ device 2540 and fed into a constant current, stimulus isolation unit. Stimulus amplitudes varied between 0 and 100 μA.

Data acquisition: a LabVIEW DAQ device 2540 was used for recording. The analog input of the DAQ device consisted of two signals recorded simultaneously at 20 kHz: the stimulus pulse train (AI1) and the cell responses (AI2). AI2 is the output of the amplifier, where the recorded cell responses were amplified 10,000 times and band-pass filtered between 300 Hz and 3 kHz.

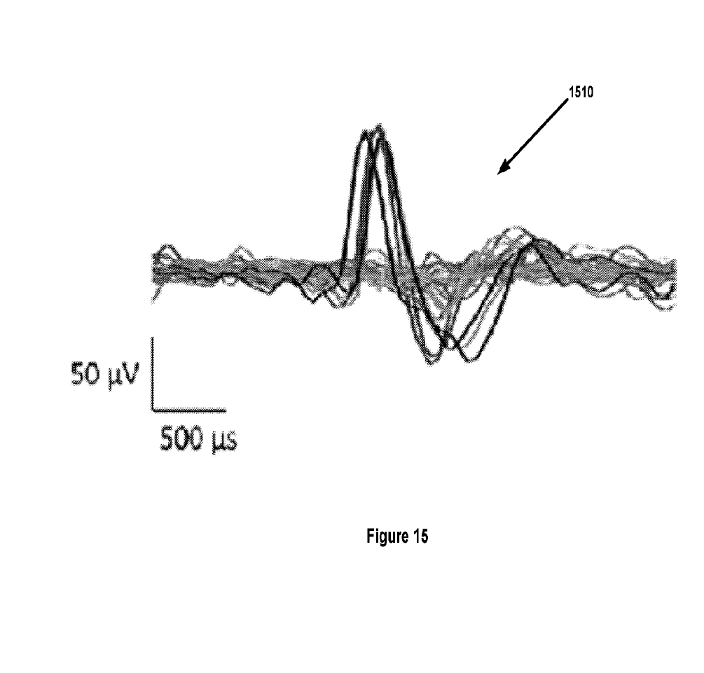

Evoked ganglion cell responses were all-or-none and are shown in FIG. 15. The evoked responses 1510 were generally time-locked to the stimulus pulses and were recorded 3 ms following the stimulation.

It should be noted that the observed latencies of several milliseconds are due to the time required for the action potential to travel along the axon and arrive at the recording site. Both the stimulating and the recording electrodes were fixed in place during stimulation of this cell and therefore any changes in the relative latencies are most likely caused by the stimulus parameters. In other words, the absolute latencies are not intrinsic properties of the cell responses but the relative changes in the latencies are.

Neural Response Model Based on Reverse-Correlation Analysis

The results in this section are for electrical stimulation. The results are based on collected experimental data.

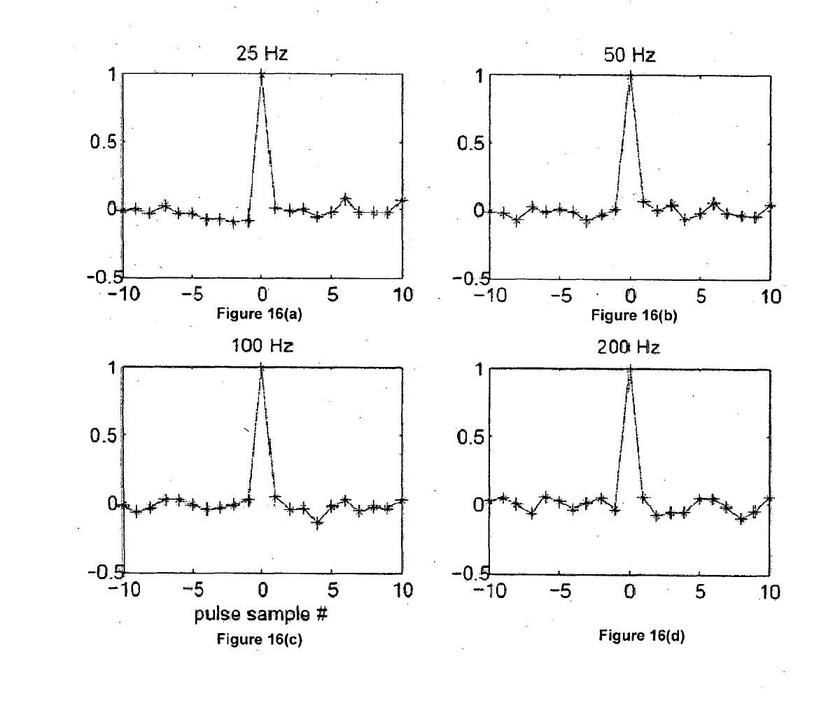

The linear impulse response kernal of the model, \vec{g}, probability of a spike to the nth pulse (a static non-linearity, G) and stochastically predicted spike train based on the reverse-correlation analysis, are given in FIGS. 16-19 below.

FIG. 16 gives the function \vec{g} for 25, 50, 100 and 200 Hz pulse rates. It is essentially a delta function with some baseline noise. It is possible that with more data and reduced baseline noise some low amplitude, less trivial aspects of the form of the function will be uncovered (from noise) and give some additional dynamics.

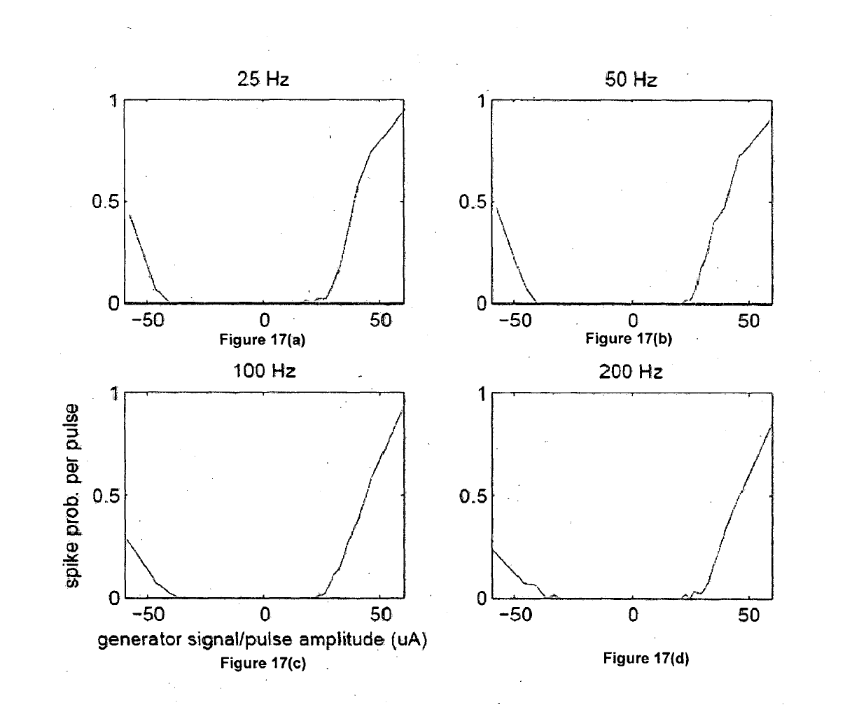

FIG. 17 gives the static nonlinearity G for 25, 50, 100 and 200 Hz pulse rates. In this case, it converts a pulse amplitude to a spike probability. It is double-sided because both cathodic first and anodic first stimulation cause spikes. The threshold is lower for cathodic first stimulation.

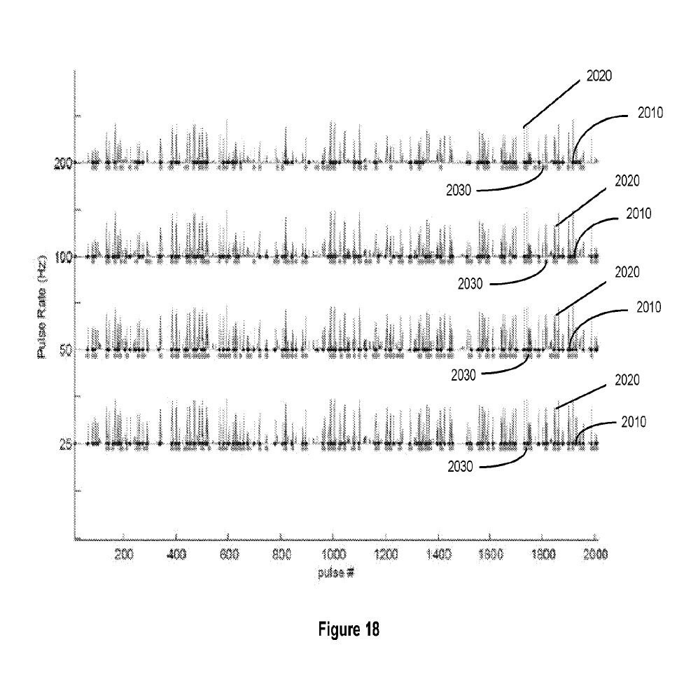

FIG. 18 gives the model predictions vs. recorded spike trains for 25, 50, 100 and 200 Hz pulse rates. For each frequency, dots 2010 indicate an experimentally recorded spike, lines 2020 give the predicted spike probability for that pulse and dots 2030 (below dots 2010) give a stochastically predicted spike train based on the spike probability.

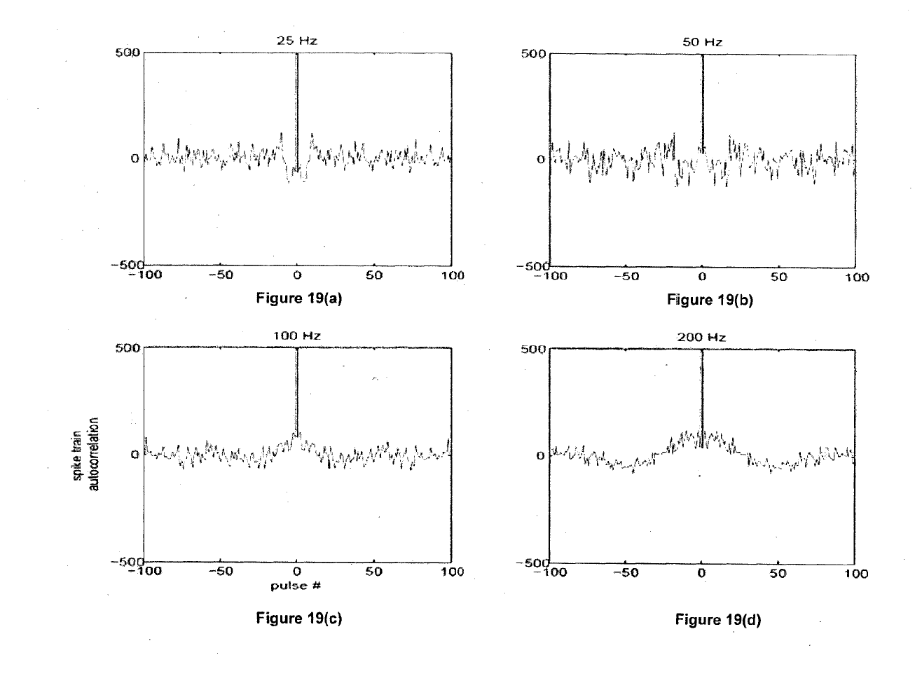

FIG. 19 gives the auto-correlation function of the recorded spike trains for 25, 50, 100 and 200 Hz pulse rates. While this is not part of the reverse-correlation analysis model, it is useful to consider because it indicates there may be some dynamics that the model is not capturing. Given the stimulus was white noise and that each pulse had a certain probability of causing a spike, independent of the other pulses, then the auto-correlation function should be a delta function on a baseline of noise. This appears not to be the case for the 200 Hz pulse train. Instead, there appears to be some non-zero correlation around the origin. This is consistent with the way that the recorded spikes tend to clump together more than the predicted spikes in FIG. 18 for 200 Hz.

Based on the analysis above, we found that using reverse correlation method we were able to reproduce the experimentally recorded spike train with good accuracy, however unable to reproduce an effect of spike clamping.

Spike History Model

The results in this section are for electrical stimulation. The results are based on collected experimental data.

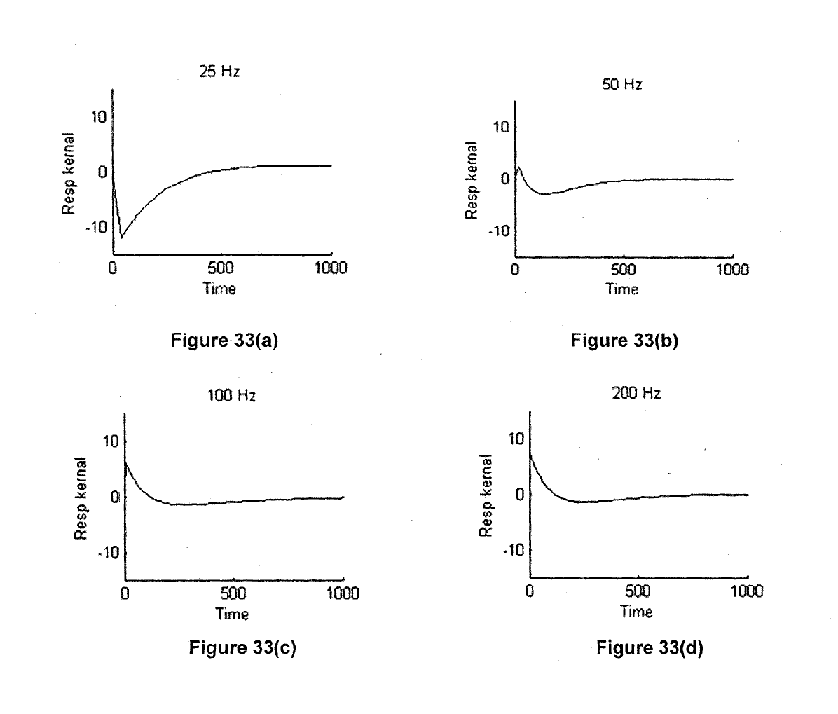

FIG. 33 gives the response kernal h for 25, 50, 100 and 200 Hz pulse rates. The function has different time constants and amplitudes for different frequencies of stimulation.

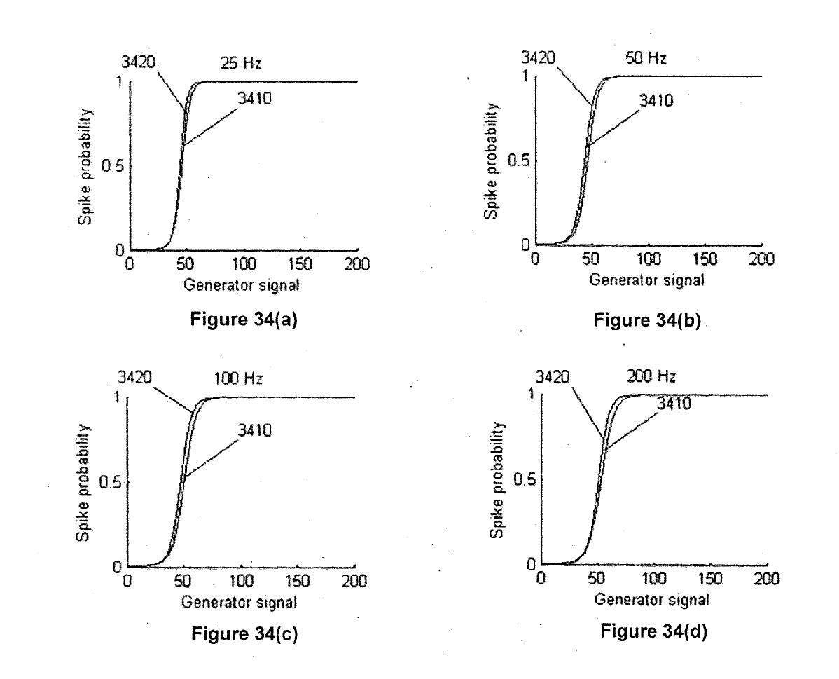

FIG. 34 gives the static nonlinearity G using spike history model with the response kernal h·\vec{r}_{[t-t}_{₁}_{, t-1]} (3410) and without the response kernal h·\vec{r}_{[t-t}_{₁}_{, t-1]}=0) (3420) for 25, 50, 100 and 200 Hz pulse rates. The function is a sigmoid, it shows a slightly lower threshold for spiking using spike history model.

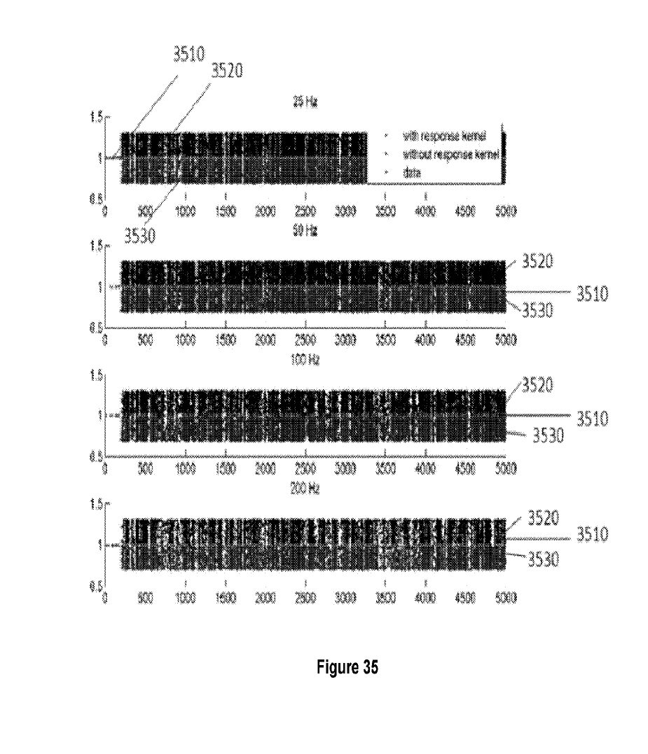

FIG. 35 gives the model predictions vs. recorded spike trains 25, 50, 100 and 200 Hz pulse rates. For each frequency, dots 3510 indicate an experimentally recorded spike, dots 3520 give a stochastically predicted spike train based on spike history model with response kernal h·\vec{r}_{[t-t}_{₁}_{, t-1]} (for multiple simulation runs), and dots 3530 give a stochastically predicted spike train based on spike history model without response kernel h·\vec{r}_{[t-t}_{₁}_{, t-1]}=0) (for multiple simulation runs).

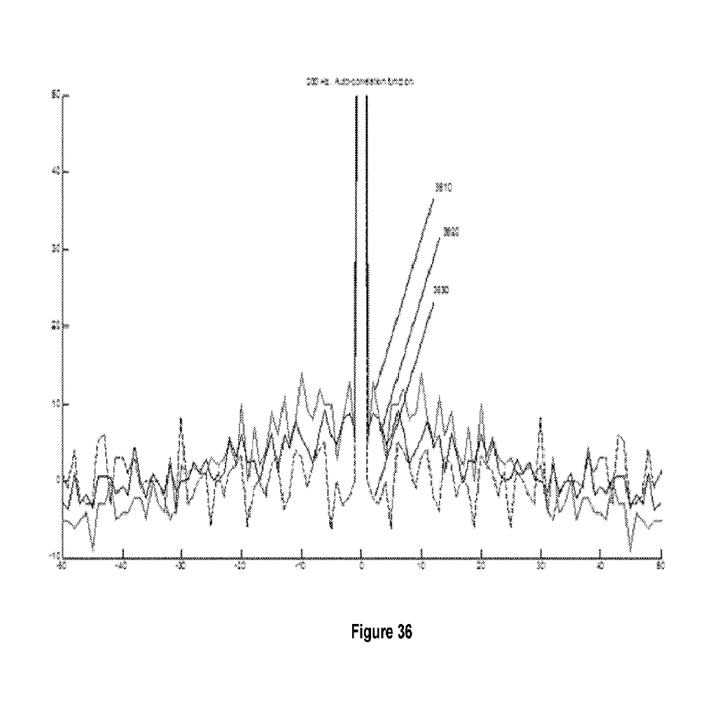

FIG. 36 gives the auto-correlation function of the recorded spike trains for 200 Hz stimulation for data (3610), the auto-correlation function of the spike history model with response kernal h·\vec{r}_{[t-t}_{₁}_{, t-1]} (3620) and the auto-correlation function of the spike history model without response kernel (h·\vec{r}_{[t-t}_{₁}_{, t-1]}=0) (3630). FIG. 36 shows that the auto-correlation function of the model with the response kernal approximates the auto-correlation function of the experimental data with better accuracy than the auto-correlation function of the model without the response kernal.

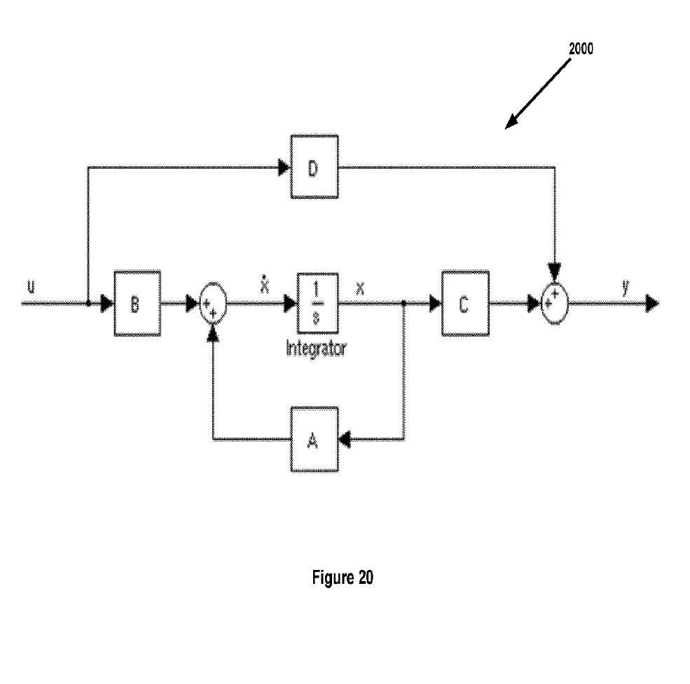

A state-space representation 2000 of the feedback system is shown in FIG. 20.

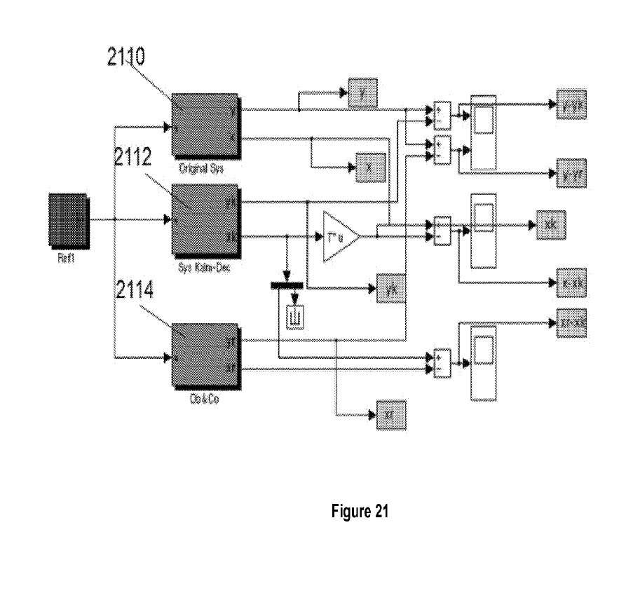

FIG. 21 provides a block-diagram for a comparison of the output of the system of equation (10), the system in Kalman decomposition of equation (12) and reduced system of equation (14).

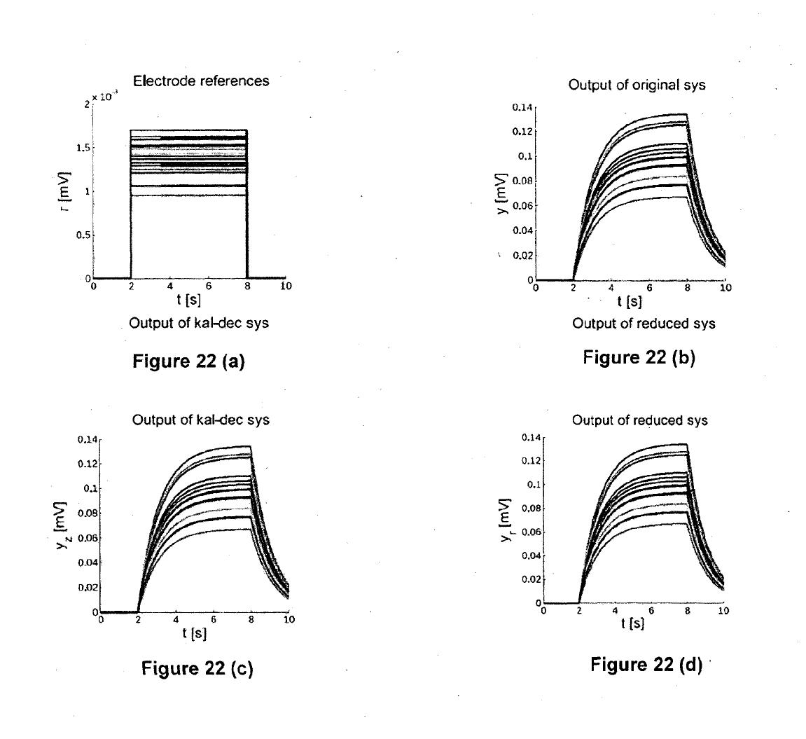

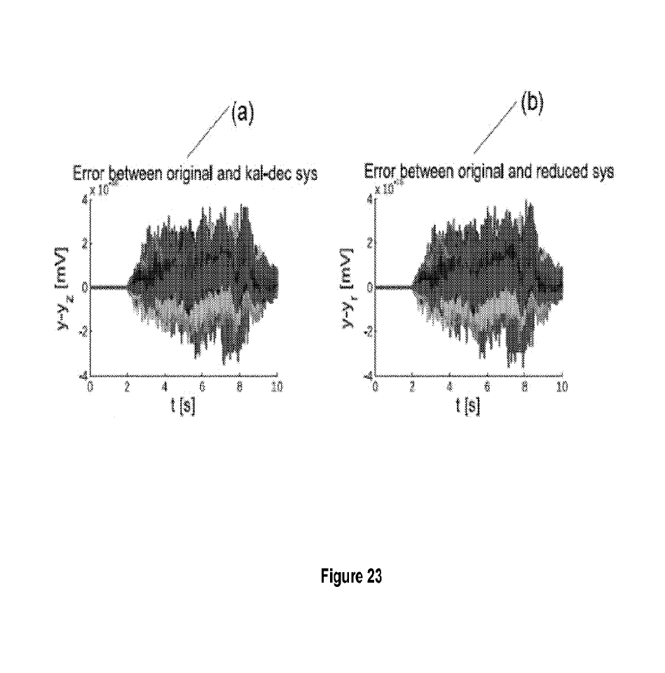

FIG. 22 shows the original system 2110 of equation (10), the system 2112 in Kalman decomposition of equation (12) and reduced system 2114 of equation (14) respond in the same way to a reference signal 2140. The systems are simulated for a number of inputs. FIGS. 22a to 22c provide a further comparison of the output of the original system of equation (10) (FIG. 22a), the system in Kalman decomposition of equation (12) (FIG. 22b) and reduced system of equation (14) (FIG. 22c). FIG. 23a shows the error between the output of the original system and system in Kalman decomposition form. FIG. 23b shows the error between the output of the original and reduced systems.

As observed in FIG. 22,

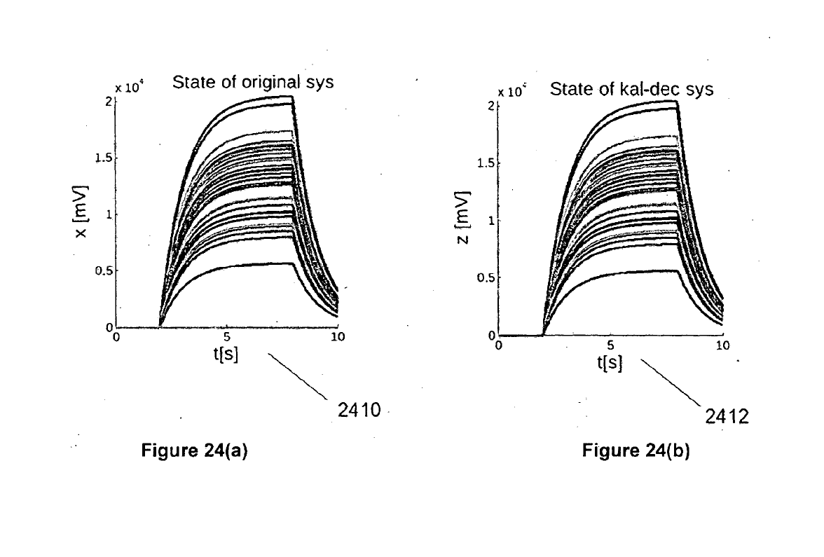

Y≅Y_{z}≅Yᵣ,

where y is the output 3110 of the original system (10), y_{z} is the output 3120 of the system in Kalman decomposition form (12) and yᵣ is the output 3130 of the reduced system (14). Therefore, we can conclude that the state of the system in Kalman decomposition form approximates the state of the original system with good approximation. The state evolutions of the original system 2410 and the system in Kalman decomposition form 2412 are given in FIG. 24.

In the above description certain steps are described as being carried out by a processor, it will be appreciated that such steps will often require a number of sub-steps to be carried out for the steps to be implemented electronically, for example due to hardware or programming limitations.

In some embodiments, the method may be embodied in program code. The program code could be supplied in a number of ways, for example on a tangible computer readable storage medium, such as a disc or a memory device, e.g. an EEPROM, (for example, that could replace part of a memory of a prosthetic apparatus) or as a data signal (for example, by downloading it into a memory of the stimulus generator from a server). Further different parts of the program code can be executed by different parts of the apparatus and hence by different processors.

Herein the term “processor” is used to refer generically to any device that can generate and process digital signals. However, typical embodiments will use a digital signal processor optimized for the needs of digital signal processing. Persons skilled in the art, will appreciate that program code provides a series of instructions executable by a processor.

It will be understood to persons skilled in the art of the invention that many modifications maybe made without departing from the spirit and scope of the invention, in particular it will be apparent that certain features of embodiments of the invention can be employed to form further embodiments.

It is to be understood that, if any prior art is referred to herein, such reference does not constitute an admission that the prior art forms a part of the common general knowledge in the art in any country.

In the claims that follow and in the preceding description of the invention, except where the context requires otherwise due to express language or necessary implication, the word “comprise” or variations such as “comprises” or “comprising” is used in an inclusive sense, i.e. to specify the presence of the stated features but not to preclude the presence or addition of further features in various embodiments of the invention.

1. A stimulus generator arranged to generate an electrical stimulus to be applied by one or more electrodes to stimulate one or more neurons, wherein in order to generate the stimulus, the stimulus generator:
implements one or more neural models defining estimated dynamic behaviour of the one or more neurons; and
employs a feedback signal indicative of at least one modelled response of the one or more neurons to at least one previously applied stimulus.

2. The stimulus generator as claimed in claim 1, wherein the neural model is based on a spike triggered average response of neurons to stimulus and incorporates a spike history response.

3. The stimulus generator as claimed in claim 1, wherein an output of the neural model depends on at least one response of one or more neurons in a time period preceding a time period in which the current stimulus is being generated.

4. The stimulus generator as claimed in claim 1, wherein the output of the neural model depends on a stimulus applied in a time period preceding a time period in which the current stimulus is being generated.

5. The stimulus generator as claimed in claim 1, comprising a reference signal generator arranged to generate a reference signal to be employed in generating the stimulus.

6. The stimulus generator as claimed in claim 5, wherein a neural modelling component of the stimulus generator implements the neural model defining estimated dynamic behaviour of the one or more neurons, and wherein the reference signal generator also implements a neural model defining estimated dynamic behaviour of the one or more neurons in response to sensory stimulus.

7. The stimulus generator as claimed in claim 6, wherein the sensory stimulus is a visual stimulus.

8. The stimulus generator as claimed in claim 5, wherein the neural model is based on a spike-triggered average response of neurons to stimulus.

9. The stimulus generator as claimed in claim 5, wherein a neural modelling component of the stimulus generator implements the neural model defining estimated dynamic behaviour of the one or more neurons, and wherein the reference signal generator implements the same type of neural model to generate the reference signal.

10. The stimulus generator as claimed in claim 5, wherein a neural modelling component of the stimulus generator implements the neural model defining estimated dynamic behaviour of the one or more neurons, and wherein the reference signal generator implements a different type of neural model to generate the reference signal.

11. The stimulus generator as claimed in claim 1, wherein the estimated dynamic behaviour is based on estimated dynamic behaviour of normally functioning neurons.

12. The stimulus generator as claimed in claim 11, comprising a reference signal generator arranged to generate a reference signal to be employed in generating the stimulus, the reference signal corresponding to the expected response of normally functioning neurons.

13. The stimulus generator as claimed in claim 8, wherein a reference signal is calculated for each electrode.

14. The stimulus generator as claimed in claim 1, comprising a static observer that provides an estimate of the internal state of the model on the basis of the stimulus and measured response of one or more neurons.

15. The stimulus generator as claimed in claim 1, comprising a dynamic observer that provides an estimate of the internal state of the model on the basis of the stimulus and measured response of one or more neurons.

16. The stimulus generator as claimed in claim 1, wherein the feedback signal is based on at least one prior response of at least one of the one or more neurons to a previously applied stimulus.

17. The stimulus generator as claimed in claim 1, wherein the feedback signal is based on at least one prior response of one or more related neurons to a previously applied stimulus.

18. A stimulus generator arranged to generate an electrical stimulus to be applied by one or more electrodes to stimulate one or more neurons, the stimulus generator comprising:
a controller arranged to generate the electrical stimulus; and
a neural modelling component that implements one or more neural models defining estimated dynamic behaviour of the one or more neurons to generate a modelled response indicative of at least one response of the one or more neurons to the generated stimulus; and
wherein the stimulus generator is arranged such that a feedback signal is provided to the controller based on the modelled response, and the controller generates a subsequent electrical stimulus based on the feedback signal.

19. The stimulus generator as claimed in claim 18, comprising an observer that receives and processes the modelled response and the generated electrical stimulus in order to generate the feedback signal.

20. A neuroprosthetic apparatus comprising the stimulus generator of claim 1 and a plurality of electrodes for applying the generated stimulus and measuring the response of one or more neurons.

21. The neuroprosthetic apparatus as claimed in claim 20 wherein the plurality of electrodes comprise separate stimulation and measurement electrodes.

22. The neuroprosthetic apparatus as claimed in claim 20, comprising at least one input device for obtaining an external stimulus.

23. A stimulation method for generating an electrical stimulus to be applied in order to stimulate one or more neurons, the method comprising implementing a neural response model defining estimated dynamics of one or more neurons, and adjusting the stimulus to be applied based on a feedback signal indicative of at least one prior response of the one or more neurons to at least one previously applied stimulus.

24. A stimulation method for generating an electrical stimulus to be applied by one or more electrodes, to stimulate one or more neurons, the method comprising:
using a neural response model defining estimated dynamics of one or more neurons to determine at least one modelled response of the one or more neurons to at least one previously applied electrical stimulus; and
generating a current electrical stimulus to be applied based on a feedback signal indicative of the at least one modelled prior response.

25. A non-transitory computer readable medium comprising code that, when executed, causes an apparatus to perform a process comprising:
implementing a neural response model defining estimated dynamics of one or more neurons; and
adjusting the stimulus to be applied based on a feedback signal indicative of at least one prior response of the one or more neurons to at least one previously applied stimulus.

26. (canceled)
